(19) United States
(12) Patent Application Publication
(10) Pub. No.: US 2010/0011842 A1
(43) Pub. Date: Jan. 21, 2010

(54) BIOCHEMICAL ASSAY METHODS

(75) Inventors: Christopher Kevin Hoyle, Philadelphia, PA (US); Theresa Jane Pell, Philadelphia, PA (US); Stephanie Yuk Fan Wong Hawkes, Philadelphia, PA (US); Brian Herbert Warrington, Hertfordshire (GB)

Correspondence Address: EKSIGENT TECHNOLOGIES, LLC;c/o SHELDON MAK ROSE & ANDERSON, 100 Corson Street, Third Floor, PASADENA, CA, 91103-3842, US

(73) Assignee: EKSIGENT TECHNOLOGIES, LLC, Dublin, CA (US)

(21) Appl. No.: 11/719,533

(22) PCT Filed: Aug. 10, 2006
(86) PCT No.: PCT/US06/31170

§ 371 Date: Sep. 2, 2009

Related U.S. Application Data
(60) Provisional application No. 60/707,374, filed on Aug. 11, 2005.

(51) Int. Cl. G01N 33/00

(52) U.S. Cl. 73/61.41

(57) ABSTRACT

Biochemical Assay Methods. There is described a method for determining a value that allows the effect that a compound has on a target to be compared with the effect that another compound has on the target, which method comprises adding the compound, at a concentration which continuously varies with time, to a flow of the target. The method can be carried out using a microfluidic system.

### CROSS-REFERENCE TO RELATED APPLICATIONS

This application claims the benefit of U.S. Patent Application Ser. No. 60/707,374, filed Aug. 11, 2005, the disclosure of which is incorporated herein by reference in its entirety. The disclosures of the following U.S. Provisional Applications, commonly owned and simultaneously filed Aug. 11, 2005, are all incorporated by reference in their entirety: U.S. Provisional Application entitled MICROFLUIDIC APPARATUS AND METHOD FOR SAMPLE PREPARATION AND ANALYSIS, U.S. Provisional Application No. 60/707,373 (Attorney Docket No. 447/99/2/1); U.S. Provisional Application entitled APPARATUS AND METHOD FOR HANDLING FLUIDS AT NANO-SCALE RATES, U.S. Provisional Application No. 60/707,421 (Attorney Docket No. 447/99/2/2); U.S. Provisional Application entitled MICROFLUIDIC BASED APPARATUS AND METHOD FOR THERMAL REGULATION AND NOISE REDUCTION, U.S. Provisional Application No. 60/707,330 (Attorney Docket No. 447/99/2/3); U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4); U.S. Provisional Application entitled METHODS AND APPARATUSES FOR GENERATING A SEAL BETWEEN A CONDUIT AND A RESERVOIR WELL, U.S. Provisional Application No. 60/707,286 (Attorney Docket No. 447/99/2/5); U.S. Provisional Application entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); U.S. Provisional Application entitled MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2); U.S. Provisional Application entitled MICROFLUIDIC SYSTEMS, DEVICES AND METHODS FOR REDUCING BACKGROUND AUTOFLUORESCENCE AND THE EFFECTS THEREOF, U.S. Provisional Application No. 60/707,386 (Attorney Docket No. 447/99/3/3); U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2); U.S. Provisional Application entitled METHODS FOR CHARACTERIZING BIOLOGICAL MOLECULE MODULATORS, U.S. Provisional Application No. 60/707,328 (Attorney Docket No. 447/99/5/1); U.S. Provisional Application entitled METHODS FOR MEASURING BIOCHEMICAL REACTIONS, U.S. Provisional Application No. 60/707,370 (Attorney Docket No. 447/99/5/2); and U.S. Provisional Application entitled METHODS AND APPARATUSES FOR REDUCING EFFECTS OF MOLECULE ADSORPTION WITHIN MICROFLUIDIC CHANNELS, U.S. Provisional Application No. 60/707,366 (Attorney Docket No. 447/99/8); U.S. Provisional Application entitled PLASTIC SURFACES AND APPARATUSES FOR REDUCED ADSORPTION OF SOLUTES AND METHODS OF PREPARING THE SAME, U.S. Provisional Application No. 60/707,288 (Attorney Docket No. 447/99/9); U.S. Provisional Application entitled FLOW REACTOR METHOD AND APPARATUS, U.S. Provisional Application No. 60/707,233 (Attorney Docket No. 447/99/11); and U.S. Provisional Application entitled MICROFLUIDIC SYSTEM AND METHODS, U.S. Provisional Application No. 60/707,384 (Attorney Docket No. 447/99/12).

### TECHNICAL FIELD

The subject matter disclosed herein relates to biochemical assays and, in particular, to the assessment of the effect that a compound (e.g. an inhibitor or an activator) has on the activity of a target. More specifically, this subject matter disclosed herein relates to the determination of properties of inhibitors and/or activators and, in particular, inhibitory concentration values (ICₓ) and/or effective concentration values (ECₓ), where x is a percentage of target activity.

The documents referred to in this specification are all incorporated fully herein by reference.

### BACKGROUND ART

Modulation of the activity of biological targets, such as proteins and, in particular, enzymes and receptors, by specific small molecules and ions is important because targets play a major role in control mechanisms in biological systems. Furthermore, many drugs and toxic agents act by inhibiting or activating these targets. Assessing the interaction between targets and compounds usually involves determining a value that allows the effect that a particular compound has on a target to be compared with the effect that another compound has, or other compounds have, on the target. Such assessments (and, in particular, the assessment of inhibitors or activators) typically involve the measurement of ICₓ or ECₓ values, respectively, where x is a percentage of target activity. For ICₓ values, x is the percentage inhibition of the target. Thus, when x is 90, the target activity is 10%. For ECₓ values, x is the percentage of the activity of the target. Useful parameters are when x is 50, i.e. IC₅₀ or EC₅₀ values. Although quoted less commonly, parameters for other values of x (e.g. IC₃₀, EC₃₀, IC₉₀ or EC₉₀ values) may also be useful. The skilled person will appreciate that, in some situations, the values of x are not confined to the range 0 to 100. This is because it may be possible to activate a target beyond what is considered as being 100% activity and, conversely, it may be possible to reduce the target activity beyond a 0% basal activity.

The ICₓ value of a compound is obtained from its inhibition curve (a plot of concentration of inhibitor on the x-axis and percentage inhibition on the y-axis) by identifying the inhibitor concentration that produces x% inhibition of the target. The ECₓ value of a compound is obtained from its activation curve (a plot of concentration of activator on the x-axis and percentage of maximum activation on the y-axis) by identifying the activator concentration that produces x% of the activation of the target. The inhibition or activation curves are typically three or four parameter logistic model based curves.

The skilled person will be aware of other values that are related to ICₓ or ECₓ values. Such values may allow the effect that a compound has on a target to be compared with the effect that another compound has, or other compounds have, on the target. One value that is related to ICₓ or ECₓ values is the inhibition constant, Kᵢ, which is independent of substrate concentration, unlike an ICₓ value which may change with substrate concentration. Kᵢ values are related to IC₅₀ values by the Cheng-Prussof relationship for competitive, non-competitive and uncompetitive inhibitors, as described at pages 285-286, of the publication titled Enzymes: A Practical Introduction to Structure, Mechanism and Data Analysis Copeland R A (ed): (Wiley-VCH, 2nd Edition, 2000 1) (hereinafter, “the Copeland Publication”). ICₓ values may be measured at different substrate concentrations and then used to determine the Kᵢ value. Kᵢ values are often preferred for describing inhibition of a target by an inhibitor. However, their determination requires more experimental data and so ICₓ values are more commonly quoted. For activators, K_{A} values are quoted rather than Kᵢ values. K_{d} values may also be of use.

In a traditional biological assay, the ICₓ value or ECₓ value of a compound is determined by performing multiple discrete (and often serial) dilutions of the inhibitor or activator. This approach is described in pages 282-287, of the Copeland Publication; Gottlin E B, Benson R E, Conary S, Antonio B, Duke K, Payne E S, Ashraf S S, Christensen D J: High-throughput screen for inhibitors of 1-deoxy-D-xylulose 5-phosphate reductoisomerase by surrogate ligand competition (J. Biomol. Screening (2003); 8 (3): 332-339); Andrisano V, Bartolini M, Gotti R, Cavrini V, Felix G: Determination of inhibitors' potency (IC₅₀) by a direct high-performance liquid chromatographic method on an immobilised acetylcholinesterase column (J. Chromatography (2001); 753: 375-383); and European Patent No. 1 164 200 A to Ekins et al., applicant Pfizer Products Inc. Thus, every compound for which an ICₓ value or ECₓ value is determined must be assayed at every concentration in the dilution series. Consequently, the use of such techniques may be time-consuming and require large volumes of compound, target and other reagents. Furthermore, the use of such techniques may involve interpolation between two points on the inhibition or activation curve in order to determine the location of the ICₓ or ECₓ value, which may compromise the accuracy of the determination.

The most common method of deriving the ICₓ/ECₓ of an inhibitor/activator involves diluting the inhibitor/activator a number of times to provide a discontinuous dilution range. This is typically done by diluting the inhibitor/activator ten times (so as to provide eleven different concentrations) in 3-fold dilution steps to provide a discontinuous dilution range that provides a dilution factor of 3¹⁰ (i.e. the concentration at the end of the range is 3¹⁰ times less than the concentration at the beginning of the range), which is approximately equivalent to providing a range spanning a 59,000-fold dilution. Experiments that involve diluting the inhibitor/activator in 2-fold steps ten times, to provide a dilution factor of 2¹⁰, are also common.

Recent developments in the field of microfluidic assay systems have been described in the publication titled Comparison of On-Chip and Off-Chip Microfluidic Kinase Assay Formats, by Dunne J, Reardon H, Trinh V, Li E, Farianas J: (Assay Drug Dev Tech (2004); 2 (2): 121-129) (hereinafter, “the Dunne Publication”) and the publication titled A Generic Assay for Phosphate-Consuming or -Releasing Enzymes Coupled On-Line to Liquid Chromatography for Lead Finding in Natural Products, by Schenk T, Appels N M G M, van Elswijk D A, Irth H, Tjaden U R and van der Greef J: (Analytical Biochemistry (2003); 316: 118-126) (hereinafter, “the Schenk Publication”). These systems involve the use of continuously flowing assay reagents into which the compound (i.e. potential inhibitor or activator) is introduced at discrete concentrations.

In the Dunne Publication and the Schenk Publication, there is again the disadvantage that interpolation is required to obtain the IC₅₀ or EC₅₀ values. However, the main disadvantage with the methodologies described in these publications is that the user must have an awareness of the approximate IC₅₀ or EC₅₀ value before he begins the experiment. In other words, it would not be practical to use these techniques to determine the ICₓ or ECₓ of a compound when its ICₓ or ECₓ value is unknown. This is because, as mentioned above, when an ICₓ or ECₓ is unknown, an assay will typically require diluting the compound ten times in 3-fold dilution steps to provide a discontinuous dilution range that provides a dilution factor of 3¹⁰. Even using continuously flowing assay reagents to produce a continuous concentration gradient, it is not possible to provide dilution factors this large. Therefore, such continuous flow techniques simply cannot probe the large concentration spans required to determine ICₓ or ECₓ values of a number of structurally dissimilar compounds, for which the ICₓ or ECₓ values are unknown.

The publication titled A Continuous-Flow System for High-Precision Kinetics Using Small Volumes, by Zhou X, Medhekar R, Toney M: (Anal. Chem. (2003); 75: 3681-3687) describes a continuous flow system that provides high precision data. However, as described above, the technique relies on a prior awareness of the parameter under investigation. Indeed, the experiments described do not determine values that are previously unknown, but probe the kinetics and mechanisms of known reactions using, for example, proton inventory experiments. Furthermore, there is no mention of using the techniques disclosed therein to study inhibitors or activators.

Similarly, the publication titled Kinetic Isotope Effects for Dialkyglycine Decarboxylase Via a High-Precision Continuous-Flow Method, by Zhou X, Toney M: (J. Am. Chem. Soc. (1998); 120: 13282-13283) and the publication titled Inhibition Patterns Obtained Where an Inhibitor is Present in Constant Proportion to Variable Substrate, by Cleland W W, Gross M, Folk J E: disclose methods for performing high-resolution kinetic studies to probe mechanistic detail, rather than a method of determining parameters that are previously unknown.

Clearly, therefore, there are limits to the application of such techniques in drug discovery, where novel compounds, such as potential inhibitors or activators, which have unknown ICₓ or ECₓ values, are synthesised.

Miniaturisation of laboratory processes is considered to be of key importance in the future of biological science and chemistry. This is because chemical and biological reactions happen faster at microscale as a result of low diffusion distances and efficient heat transfer. Furthermore, the use of miniaturised, continuously flowing assay reagents reduces the wastage of reagents, enables the use drug targets of low availability, increases experimental speed and provides a real-time system for self-optimisation. In this regard, European Patent No. 1 336 432 A to Gilligan et al., applicant Syrris Ltd., (hereinafter, “EP '432”) is of relevance. This discloses a method of optimising a reaction in a microreactor. Two reaction fluids are supplied to a microchannel and their relative proportions are varied in a controlled manner. A sensor then monitors a reaction characteristic and determines the relative proportion of the fluids which optimises the yield of the reaction product. The total flow rate can also be varied at the optimum relative proportion in order to determine the maximum overall flow rate at which completion of the reactions occurs. More specifically, EP '432 relates to a microreactor comprising a reaction channel.

The microreactor also comprises:

a first reaction fluid supply system comprising a reservoir of a first reaction fluid, a means to deliver a controlled amount of the first reaction fluid to flow through the reaction channel, and means to monitor the flow of the first reaction fluid into the reaction channel;

a second reaction fluid supply system comprising a reservoir of a second reaction fluid, a means to deliver a controlled amount of the second reaction fluid to flow through the reaction channel, and means to monitor the flow of the second reaction fluid into the reaction channel;

a sensor to monitor a characteristic produced when the first and second reaction fluids react; and

a controller which receives inputs from the means to monitor the flows of the first and second reaction fluids and from the sensor and controls the means to deliver the controlled amounts of first and second reaction fluids to the reaction channel, wherein the controller is arranged to vary across a range of values the relative proportions of the first and second reaction fluids fed to the reaction channel and to detect the relative proportions of the first and second reaction fluids which optimise the yield of the reaction product.

Glass, quartz and plastic chips with microchannels having cross-sectional diameters between 5 and 500 μm have been used to perform chemical and biological experiments and assays. In such chips, the interconnected channel network allows nanolitres of fluids to be precisely metered and transported. Pressure-driven flow, electrokinetic flow or a combination of the two moves the fluids along the channels. Reagents are introduced into the channels either from wells on the chip or from capillaries attached to the chips.

The Dunne Publication describes an assay in which an enzymatic reaction takes place in the microchannels of a chip. This paper also reports a related method where the enzymatic reaction takes place on a microtitre plate. A commercially available microfluidics platform is used. In the on-chip method, sample inhibitors are introduced into the microchannels of the chip, mixed with enzyme and substrates, which are also introduced into the microchannels of the chip, and allowed to react on the chip. The amount of product generated is quantified by electrophoretic separation of the reaction mixture. It is disclosed that the small dimensions of the microchannels allow unique capabilities not easily achievable in microtitre plates, including fast mixing, very small reaction volumes (tens of nanolitres), rapid temperature changes, precise reagent addition and electrophoretic separations. In the on-chip methods disclosed in this paper, the inhibitor is introduced into the microchannels of the chip at a fixed concentration. Using such a method, it is possible to determine the percentage inhibition, in relation to a particular enzyme and substrate, for a series of discrete concentrations of inhibitor and thus determine the IC₅₀ value.

The Dunne Publication does not disclose a method for determining IC₅₀ values, in which the concentration of the inhibitor varies continuously. Rather, the inhibition curves presented in this paper are constructed using discrete data.

Even if the inhibitor concentration were varied continuously, it would not be possible to use the technique of the Dunne Publication to determine an ICₓ value, when the ICₓ value is unknown prior to the experiment. This is because a sufficiently large concentration range could not be achieved. There is still a need to develop a technique in which ICₓ/ECₓ values (and other values that allow the effect that a compound has on a target to be compared with the effect that another compound has, or other compounds have, on the target) can be determined when they are unknown prior to the experiment. Put more broadly, there is still a need to develop an improved technique for determining a value that allows the effect that a compound has on a target to be compared with the effect that another compound has on the target.

Furthermore, there is a desire for techniques that provide more accurate results than those known in the art, further reduce the wastage of reagents and avoid problems with sample storage and waste production. It is therefore an aim of the subject matter disclosed herein to provide an improved method for determining values that allow the effect that a compound has on a target to be compared with the effect that another compound has on the same target. In particular, it is an aim of the subject matter disclosed herein to provide an improved method for determining ICₓ and ECₓ values. As explained above, it is also an aim to provide a method that can be used to determine values (e.g. ICₓ or ECₓ values), using continuous flow techniques, even when the values are unknown.

### BRIEF DESCRIPTION OF THE DRAWINGS

FIG. 1 is a schematic view of a system for continuously varying compound concentration with time;

FIG. 2 is a schematic diagram of an apparatus adapted to be used to determine a value that allows the effect that a compound has on a target to be compared with the effect that another compound has on the target;

FIG. 3 is a photograph of a fluorescence intensity microbiochemistry platform suitable for performing the methods of the subject matter disclosed herein;

FIG. 4 is a photograph of an incubator housing and an x, y, z positioning stage, suitable for use in conjunction with the subject matter disclosed herein;

FIG. 5 is a photograph of a microchannel device in an incubator, suitable for use in conjunction with the subject matter disclosed herein;

FIG. 6 is an embodiment of the subject matter disclosed herein in which overlapping concentration gradients are used to calculate the IC₅₀ value of an inhibitor;

FIG. 7 is an example of a decision-making process that could be used in the ‘triage’ approach;

FIGS. 8a and 8b are graphs of data collected using an embodiment of the subject matter disclosed herein in which adjacent concentration gradients overlap to some extent; and

FIGS. 9a and 9b are graphs of data collected using the overlapping gradient approach of the subject matter disclosed herein.

### DETAILED DESCRIPTION

According to one embodiment of the subject matter disclosed herein, there is provided a method for determining a value that allows the effect that a compound has on a target to be compared with the effect that another compound has on the target, which method comprises adding the compound, at a concentration which continuously varies with time, to a flow of the target.

The method can be used to compare the effect of a compound with the effect of a number of other compounds.

In one embodiment, the value is the ICₓ value or ECₓ value of the compound, wherein x is a percentage of the target activity. In one embodiment, the compound is an inhibitor or activator of the target. Thus, the subject matter disclosed herein may provide a method for determining ICₓ values of an inhibitor or ECₓ values of an activator, which method comprises adding the inhibitor or activator, at a concentration which continuously varies with time, to a flowing source of a target which may be inhibited or activated by the inhibitor or activator, respectively.

Alternatively, the compound may have no effect on the activity of the target, i.e. it may be inactive. Finding that a compound is inactive may be an important discovery. For example, a scientist may benefit from knowing that particular structures are inactive against a particular target. This may be because valuable information about how structure affects activity can be obtained from examining the differences in structure between an active and an inactive compound.

The value need not be an ICₓ value or an ECₓ value, but may be any value that allows the effect that a compound has on a target to be compared with the effect that another compound has on the target. Thus, the values determined for a set of compounds may allow the compounds to be ranked in order of how potently they affect the activity of the target. A series of inhibitors, activators or inactive compounds may thereby be ranked in order of potency. Furthermore, the value need not be a formally recognized scientific parameter nor need it be formally reported to the user of the method. In one embodiment, the value is in a form that cannot be readily determined by the user, e.g. an electrical or electromagnetic signal which is interpreted by a machine before being relayed to the user. For example the machine may interpret the electric or electromagnetic signals and thereby rank the compounds in order of potency such that the user is ultimately provided with a list of compounds ranked in order of potency.

The method of the subject matter disclosed herein leads to more accurate results and a considerable reduction (by 100 to 1000-fold) in wastage of compounds and other reagents over commonly used methods that involve discretely varying the concentration of a compound, e.g.

microtitre plate methods.

The reduction in reagent usage allows assays to be performed on targets that, using previous technologies, could not be assayed. This is because it has not been possible, or it has been too expensive, to synthesise the quantities of target required for use in previous technologies.

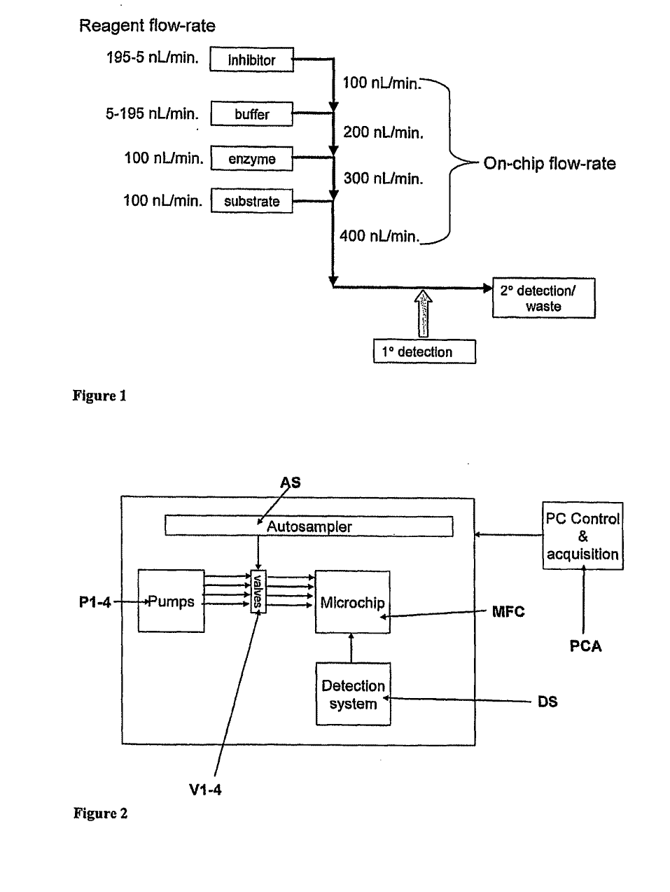

In one embodiment, the continuous variation of the compound concentration with time may be achieved by keeping the overall flow rate constant whilst changing the flow rate of the compound and/or the target. The continuous variation of the compound concentration with time can be achieved by keeping the overall flow rate constant whilst gradually changing the flow rate of the compound. A schematic example of this is given in FIG. 1 (described in greater detail below). Other examples of microfluidic systems, devices, and method for continuously varying compound concentration are disclosed in co-pending, commonly owned U.S. Provisional Application entitled MICROFLUIDIC APPARATUS AND METHOD FOR SAMPLE PREPARATION AND ANALYSIS, U.S. Provisional Application No. 60/707,373 (Attorney Docket No. 447/99/2/1); U.S. Provisional Application entitled APPARATUS AND METHOD FOR HANDLING FLUIDS AT NANO-SCALE RATES, U.S. Provisional Application No. 60/707,421 (Attorney Docket No. 447/99/2/2); U.S. Provisional Application entitled MICROFLUIDIC BASED APPARATUS AND METHOD FOR THERMAL REGULATION AND NOISE REDUCTION, U.S. Provisional Application No. 60/707,330 (Attorney Docket No. 447/99/2/3); U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4); U.S. Provisional Application entitled METHODS AND APPARATUSES FOR GENERATING A SEAL BETWEEN A CONDUIT AND A RESERVOIR WELL, U.S. Provisional Application No. 60/707,286 (Attorney Docket No. 447/99/2/5); U.S. Provisional Application entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); U.S. Provisional Application entitled MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2); U.S. Provisional Application entitled MICROFLUIDIC SYSTEMS, DEVICES AND METHODS FOR REDUCING BACKGROUND AUTOFLUORESCENCE AND THE EFFECTS THEREOF, U.S. Provisional Application No. 60/707,386 (Attorney Docket No. 447/99/3/3); U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2); U.S. Provisional Application entitled METHODS FOR CHARACTERIZING BIOLOGICAL MOLECULE MODULATORS, U.S. Provisional Application No. 60/707,328 (Attorney Docket No. 447/99/5/1); U.S. Provisional Application entitled METHODS FOR MEASURING BIOCHEMICAL REACTIONS, U.S. Provisional Application No. 60/707,370 (Attorney Docket No. 447/99/5/2); and U.S. Provisional Application entitled METHODS AND APPARATUSES FOR REDUCING EFFECTS OF MOLECULE ADSORPTION WITHIN MICROFLUIDIC CHANNELS, U.S. Provisional Application No. 60/707,366 (Attorney Docket No. 447/99/8), the contents of which are incorporated herein in their entireties.

In one embodiment, the continuous variation of the compound concentration with time is achieved by changing the flow rate of the compound whilst changing the flow rate of a component other than the target so as to keep the overall flow rate constant. For example, if the method involves a flow of a compound, a flow of a target and a flow of a third component, which third component can be a vehicle for the compound (e.g. a buffer component or 2% methanol in water), then the total flow rate of the compound and the third component may be kept constant, but the individual flow rate of the compound and the individual flow rate of the third component may be varied. In other words, the flow rate of the compound will be increased/decreased to the same extent that the flow rate of the third component is decreased/increased, respectively. Thus, the overall flow rate is kept constant.

As used herein, the term “inhibitor/activator” is shorthand for “inhibitor or activator”. This definition applies mutatis mutandis to similar notation e.g. the term “ICₓ/ECₓ” is shorthand for “ICₓ or ECₓ” and the term “inhibitors/activators/compounds” is short for “inhibitor, activator or compound”.

The method may involve more than one step of adding the compound to a flow of the target, wherein the concentration of the compound varies continuously with time during each of these steps.

The method of the subject matter disclosed herein may comprise more than one step of adding the compound to a flow of the target, wherein the concentration of the compound varies continuously with time throughout each of these steps and wherein the rate of change of compound concentration with respect to time in each step is different from the rate of change of compound concentration with respect to time in (any of) the other step(s) and/or the compound concentration range in each step is different from the compound concentration range in (any of) the other step(s). The term ‘concentration range’ is used to mean the identity of the values spanned in each range and not the difference between the concentrations at the beginning and end of the step. To further clarify the meaning of ‘concentration range’ it is easiest to consider an example. If a concentration varies from 10 units to 3 units, the ‘concentration range’ is 10 units to 3 units, whereas the difference between the concentrations at the beginning and end of the step is 7 units.

When the method comprises more than one step, within each step, the rate of change of compound concentration with time can be constant. However, in an alternative embodiment, the rate of change of compound concentration with time is not constant.

The compound concentration ranges in each step need not be the same. By using different ranges of compound concentration in each step, it is possible to study a span of concentrations that is greater than that used in one step alone. Importantly, this method is useful for determining values that allow the effect that a compound has on a target to be compared with the effect that another compound has on the same target (and, in particular, ICₓ or ECₓ values) when they are unknown.

There may be overlap between the ranges used in each step.

As mentioned above, many typical assays involve diluting the compound ten times in 3-fold dilution steps to provide a discontinuous dilution range that provides a dilution factor of 3¹⁰. Using continuous flow to produce a continuous concentration gradient, it is not possible to provide dilution factors this large.

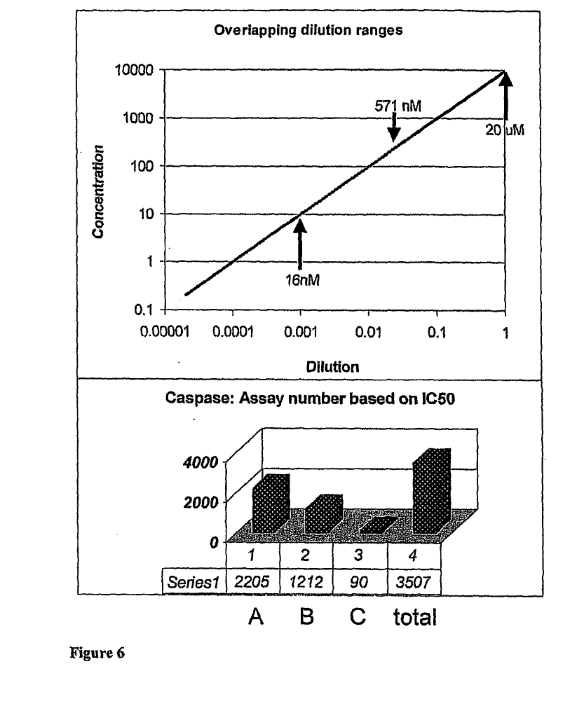

However, using an embodiment of the subject matter disclosed herein, in which there is more than one step, these problems are overcome (as illustrated in FIG. 6 and in example 1—see below). Thus, rather than relying on a prior knowledge of the value in question (e.g. the ICₓ or ECₓ value), the methodol of the subject matter disclosed herein may be used to probe a wide array of concentrations when the value in question (e.g. the ICₓ or ECₓ value) is unknown and will then act as a value/ICₓ/ECₓ“prospector”.

All the embodiments of the subject matter disclosed herein may be used to determine values (which, in one embodiment, are ICₓ or ECₓ values) that allow the effect that a compound has on a target to be compared with the effect that another compound has on the target, when said values are unknown. Embodiments of the subject matter disclosed herein may be used to determine said value of a single compound or said value of each compound in a set of compounds, which compounds may or may not be structurally similar, when said value of the single compound or said value of at least one of the set is unknown. Some embodiments of the subject matter disclosed herein are particularly useful for determining said value of each compound in a set of compounds, which compounds are structurally dissimilar, when said value of at least one, and preferably all, of the set is unknown.

As used herein, the term “unknown” means that the person using the method does not know, at the time immediately prior to using the method, the value (which, in one embodiment is an ICₓ or ECₓ value) to within 0.01%, more preferably 0.1%, more preferably 0.5%, more preferably 1%, more preferably 2%, more preferably 3%, more preferably 4%, more preferably 5%, more preferably 10%, more preferably, 15%, more preferably 20%, more preferably 30%, more preferably 50%, more preferably 75%, more preferably 97.5%, more preferably 100%, more preferably 200%, more preferably 500%, more preferably 1000% and yet more preferably 10000% of the true value. Most preferably, the term “unknown” means that the person using the method does not know the value (which, in one embodiment is an ICₓ or ECₓ value) to any extent. This may be, for example, because there is no data available for the determination of the ICₓ or ECₓ value. As used herein, the term “true value” means the value that would be determined empirically if the methods described herein were used.

In some embodiments, any of the methods of the subject matter disclosed herein described herein may be used to determine values (which, in one embodiment, are ICₓ or ECₓ values) that allow the effect that a compound has on a target to be compared with the effect that another compound has on the target, as opposed to performing experiments to investigate mechanistic detail or detailed kinetics. Thus, in one embodiment, the methods of the subject matter disclosed herein are not suitable for studying mechanistic detail or detailed kinetics. Examples of methods that are suitable for studying mechanistic detail or detailed kinetics are kinetic isotope effect investigations, proton inventory experiments, experiments to probe the identity of the rate determining step and pH dependency experiments. (Obviously, the determination of ICₓ, ECₓ values and other values as described in claim 1 does not, within the context of this specification, count as a method suitable for studying mechanistic detail or detailed kinetics). Thus, in one embodiment, the methods of the subject matter disclosed herein are not suitable for at least one of: kinetic isotope effect investigations, proton inventory experiments, experiments to probe the identity of the rate determining step and pH dependency experiments.

However, the skilled person will be aware that in alternative embodiments of the subject matter disclosed herein, the quality of data generated may be sufficiently high to allow the study of mechanistic detail and/or detailed kinetics.

In a further embodiment, the rate of change of compound concentration with time differs between each step. In an alternative embodiment, the rate of change of compound concentration with time is the same in each step.

Where the method involves more than one step of adding the compound to a flow of the target, the relative change in concentration in each step may be the same. In this context, ‘relative change’ is defined as the difference between the concentration at the beginning of the step and the end of the step, divided by the concentration at the beginning of the step. In an alternative embodiment, the relative change in concentration differs between each step.

Where the method involves more than one step of adding the compound to a flow of the target, each step of adding the compound to a flow of the target may be performed for the same length of time. In an alternative embodiment, the steps of adding the compound to a flow of the target are not all performed for the same length of time. The steps of adding the compound to a flow of the target may all be performed for different lengths of time.

The skilled artisan will appreciate that one particular combination is not a possible embodiment (except when a step is intentionally repeated to obtain duplicate results). Specifically, he will be aware that it is physically impossible to have an embodiment of the method where there is more than one step of adding the compound to a flow of the target in which:

the rate of change of compound concentration with time is the same in each step;

the relative change in concentration in each step is the same; and

each step of adding the compound to a flow of the target is performed for the same length of time.

However, one possible embodiment is a method where there is more than one step of adding the compound to a flow of the target in which:

the rate of change of compound concentration with time is the same in each step; and

the relative change in concentration in each step is the same.

Another possible embodiment is a method where there is more than one step of adding the compound to a flow of the target in which the rate of change of compound concentration with time is the same in each step, and each step of adding the compound to a flow of the target is performed for the same length of time.

Yet another possible embodiment is a method where there is more than one step of adding the compound to a flow of the target in which the relative change in concentration in each step is the same, and each step of adding the compound to a flow of the target is performed for the same length of time.

Of course, as is common practice in scientific experiments, an experiment performed using the subject matter disclosed herein may be repeated in order to provide duplicate results. Thus any step may be repeated exactly to obtain duplicate results.

In one embodiment, the concentration of the compound at the start of each step differs from the concentration at the start of (all of) the other step(s).

In another embodiment, the concentration of the compound at the end of each step differs from the concentration at the end of (all of) the other step(s).

In yet another embodiment, the concentration of the compound at the start of each step differs from the concentration at the start of (all of) the other step(s) and the concentration at the end of each step differs from the concentration at the end of (all of) the other step(s).

The subject matter disclosed herein also provides a method for determining a value that allows the effect that a compound has on a target to be compared with the effect another compound has on the target, which method comprises more than one step of:

adding the compound, at different concentrations, to a flowing source of a target,

wherein the compound concentration range in each step differs from the compound concentration range in (any of) the other step(s), and wherein x is a percentage of the maximum activity of the target.

In other words, in this embodiment, there is no need for the concentration of the compound to vary continuously with time but discrete concentrations may be used instead.

The phrase “the compound concentration range in each step differs from the compound concentration range in (any of) the other step(s)” means that the combination of discrete compound concentrations used in each step differs from the combination of discrete compound concentrations used in (any of) the other step(s). For example, if a first step uses discrete concentrations of 1, 5 and 10 units and another step uses discrete concentrations of 1, 3 and 10 units, “the compound concentration range in the first step differs from the compound concentration range in the other step”.

Preferably, the highest compound concentration, lowest compound concentration or highest and lowest compound concentrations in each step differ(s) from the highest compound concentration, lowest compound concentration or highest and lowest compound concentrations, respectively, in (any of) the other step(s). Thus, in one embodiment, the difference between the highest and lowest compound concentrations in each step differs from the difference between the highest and lowest compound concentrations in (any of) the other step(s). E.g. a first step uses discrete compound concentrations of 1, 5 and 6 units and a second step uses discrete compound concentrations of 1, 5 and 10 units. More preferably, the highest and lowest compound concentrations in each step differ from the highest and lowest compound concentrations, respectively, in (any of) the other step(s) and the difference between the highest and lowest compound concentrations in each step may or may not be the same as the difference between the highest and lowest compound concentrations in (any of) the other step(s). For example, a first step uses discrete compound concentrations of 1, 5 and 10 units and a second step uses discrete compound concentrations of 10, 15 and 20 units: the lowest and highest compound concentration in the first step differs from the lowest and highest compound concentration, respectively, in the second step. In this example, the difference between the highest and lowest compound concentrations in the first step is the same as the difference between the highest and lowest compound concentrations in the second step. The skilled person will appreciate that any number of discrete compound concentrations may be used in each step and that accuracy will be improved by using a higher number of discrete compound concentrations.

For any embodiment of the subject matter disclosed herein with more than one step of adding the compound to a flow of the target, there may be any number of steps. In one embodiment, the number of steps is 2 or more and preferably 3 or more. In another embodiment, the number of steps is from 2 to 10 and preferably the number of steps is 3.

Where the method involves more than one step of adding the compound to a flow of the target, preferably the range of compound concentrations used in each step is not the same. In such a case, there may or may not be overlap between the ranges. Preferably, there is overlap between the ranges used in at least two of the steps. More preferably, the range in each step overlaps with the range in at least one other step.

More preferably, for each step:

if there is a step immediately previous to it, its range overlaps with the range for the previous step, and

if there is a step immediately subsequent to it, its range overlaps with the range for the subsequent step.

As used herein, the phrases “step immediately previous to” and “step immediately subsequent to” mean the last step that took place before the step in question and the step that will take place straight after the step in question, respectively.

Where the method involves more than one step comprising the addition of the compound to a flow of the target, certain steps may only be performed when, within a certain other step, the compound yields a result (e.g. value) that falls within a particular range. More than one other step may be used to determine whether certain steps are performed. Thus, certain steps may only be performed when, within certain other steps, the compound yields a combination of results (e.g. values) that falls within a particular range.

In other words, where the method involves more than one step comprising the addition of the compound to a flow of the target, certain steps may only be performed when, within a certain other step or certain other steps, the compound yields an experimentally-determined result (e.g. value), or combination of experimentally-determined results (e.g. values), that falls within a particular range.

In a further embodiment, certain steps may only be performed when, within a certain other step or certain other steps:

the compound yields an ICₓ value or a combination of the ICₓ values determined individually in each step; or

the compound yields an ECₓ value or a combination of the ECₓ values determined individually in each step,

that falls within a particular range.

Preferably, the step or steps that are not performed are all those that are subsequent to the step or steps that yield the result (e.g. value) that falls within the particular range. Yet more preferably, none of the steps other than the first step are performed if, within the first step, the compound yields a result (e.g. value) that falls outside the particular range.

This increases the speed of the assays and eliminates the unnecessary testing of inactive compounds or weakly active inhibitors/activators. Thus, it is possible to assay a greater number of inhibitors/activators/compounds in a given time period.

In a further embodiment of the subject matter disclosed herein, a method for determining the values/ICₓ values/ECₓ values of more than one compound is provided, in which each compound is tested using any of the aforementioned methods. This may be defined as a high content, high throughput method and the skilled person will appreciate what is meant by a “high content, high throughput” method. In one definition, the term means that at least 1000 compounds (and, preferably at least 1300 compounds) are assayed in detail (i.e. their ICₓ values, ECₓ values or other values that allow the effect that they have on a target to be compared with the effect that other compounds have on the target are determined) in each 24 hour period.

As the “values” need not be formally reported to the user of the subject matter disclosed herein, in one embodiment, the subject matter disclosed herein ranks of a series of inhibitors/activators/compounds in order of potency and the user does not have the absolute values reported to him. Obviously, this embodiment may be used when the user is not interested in the absolute values but is concerned with their relative positions in a rank order.

The skilled person will be aware of what is meant by “a flow of the target”. In one embodiment, this term excludes microtitre plate techniques. In another embodiment, this term means that the source of the target has a net movement parallel to the sides of the channel into which it is introduced.

In another embodiment, this term means that, at the point where the compound is added to the flow of the target, the target is moving such that, at any instant, the compound is added to a target that has not yet been in contact with any compound.

The target may be present in an isolated form. Alternatively, the target may be present as part of a larger system and this may be a biological system, e.g. a cell. Therefore, the larger system, or a part thereof, may be introduced into the apparatus.

The target may be a protein. The target may be an enzyme, receptor or membrane protein. Preferably, the target is an enzyme. If the target is an enzyme, it may be a proteinase or a kinase. If the target is a receptor, it may be a nuclear receptor or a membrane-associated receptor. The target may be a domain or a sub-unit of a protein. Preferably, catalytic or binding domains are tested.

The examples describe experiments on matrix metalloproteinase 12 (MMP12), activin receptor-like kinase 5 (ALK5) and glycogen synthase 3 kinase (GSK3).

The target need not be a protein and may be a non-protein receptor. Examples of non-protein receptors include genes, polysaccharides, DNA, such as cDNA, synthetic DNA and genomic DNA, and mRNA or complexes thereof. The target may be a domain or a sub-unit of a non-protein receptor. Preferably, binding domains are tested.

Furthermore, as the target need not be a protein or, indeed, a biological species, the subject matter disclosed herein may be used in conjunction with cosmetics, consumer healthcare products, electronic devices and phosphors for television screens and other visual displays e.g. mobile phone screens and computer screens. In other words, the target can be anything which, when modulated by an inhibitor/activator, must be assayed over a concentration range spanning a large (preferably >100-fold, more preferably >1000-fold, yet more preferably >10000-fold and most preferably >50000-fold) dilution factor in order to determine the inhibitor's/activator's ICₓ or ECₓ value (or some other value as defined in claim 1) when it is unknown.

In one embodiment, it is envisaged that the target is of pharmaceutical or agrochemical interest. In another embodiment of the subject matter disclosed herein, the target is found in, or derived from, any organism, i.e. any of: a mammal, a plant, a fungus, a virus or a bacterium. Preferably, the target in found in, or derived from, a mammal and, in particular, a human.

In an alternative embodiment, the target is found in, or derived from, a bacterium. Such targets are typically used when the methods of the subject matter disclosed herein are used to search for antibiotics. In another alternative embodiment, the target is found in, or derived from, a plant.

The target may be the target in neat form or the target in a suitable vehicle e.g. the target as part of a mixture or solution or may be supported on appropriate mobile carriers (e.g. silica or polymeric beads). Alternatively, the target may comprise precursors of the target which are in equilibrium with the target itself.

The compound may be a drug to be administered to mammals and, in particular, humans.

x may, in theory, take any value. Typically, x will take any value from −100 to 200. Preferably the value of x is 50, i.e. the methods are for determining IC₅₀ or EC₅₀ values. In another embodiment, the value of x is 30, i.e. the methods are for determining IC₃₀ or EC₃₀ values. In another embodiment, the value of x is 90, i.e. the methods are for determining IC₉₀ or EC₉₀ values.

The method may comprise monitoring the activity of the target directly or indirectly.

In one embodiment of the subject matter disclosed herein, the monitoring of the target activity may comprise the use of at least one of:

Raman spectroscopy;

mass spectrometry;

electrophoresis; and

techniques that measure at least one of fluorescence intensity, time-resolved fluorescence, fluorescence lifetime, fluorescence polarization and luminescence.

The skilled person will appreciate that other techniques may be used.

The methods described above may be performed using any suitable apparatus. FIG. 2 (see below) provides a schematic diagram of a suitable apparatus.

In an embodiment of the subject matter disclosed herein, there is provided an apparatus adapted to be used to determine a value that allows the effect that a compound has on a target to be compared with the effect that another compound has on the target, which apparatus allows the addition of the compound at a concentration that continuously varies with time to a flow of the target. In a preferred embodiment, the value is the ICₓ value or ECₓ value of a compound, wherein x is a percentage of the target activity.

In other words, this apparatus allows the addition of a compound to a flow of a target and allows the concentration of the compound to be varied continuously with time during said addition.

This apparatus may perform any of the aforementioned methods.

In one embodiment, the apparatus comprises channels. Preferably, these channels have dimensions that cause flow with a Reynolds number of less than 10³ and a predominantly laminar flow regime. In an embodiment, these channels have dimensions from 1μm to 1 mm.

In an embodiment, if the apparatus has channels,

the compound flows along a channel, along which other reagents may optionally flow,

the target flows along another channel, along which other reagents may optionally flow, and

the compound is added to the target flow when said channels meet at flow junctions.

The compound may react with (and, in particular, inhibit or activate) the target when it is added to it.

Chemical and biological reactions happen faster at microscale as a result of low diffusion distances and efficient heat transfer. Further, less material is used in such reactions, resulting in less expensive and more environmentally friendly operation. Thus a microfluidic apparatus, which is also known as a “microreactor”, is preferred. Particularly preferred are microreactors in which the reaction takes place on a small reaction “chip”. Microfluidic systems are currently available for a number of applications in the biology field, for example DNA sequencing on a chip. Such systems are designed to carry out one or a series of biochemical reactions that are well understood and have known outcomes. Glass or plastic chips may be used. However, glass chips generally avoid problems associated with melting and substances that have LogD values greater than 2.

The term “microreactor” and the associated term “microchannel” are believed to be terms which are clearly understood in the art. The terms are best understood functionally as relating to reactors/channels which are sufficiently small that diffusional mixing predominates and efficient heat transfer occurs, resulting in optimal reaction conditions in the microchannel. A microreactor is a microfluidic device used for carrying out chemical reactions. In a typical microreactor, chemical reagents flow along microchannels and react when combined at flow junctions.

The dimensions should be sufficiently small that they cause a flow with a low Reynolds number (<10³, preferably <10², and more preferably <10) and a predominantly laminar flow regime. In a laminar flow regime, diffusional mixing defines the rate of chemical reactions. The rate of diffusion between two chemical reagents in a microreactor is defined by Fick's law. In this sense, “predominantly” means that more than 60%, preferably more than 80%, and most preferably more than 90%, by volume of the fluid has a laminar flow regime.

Generally, the reactor/channel should have, in cross-section, a maximum cross-sectional dimension of 5 μm to 500 mm, preferably 5 to 250 μm and more preferably 10 to 100 μm. However, it is possible to envisage a channel which has a long thin cross-section having a dimension greater than imm, but which still operates as a microreactor as it is small in other dimensions. Therefore, it might be more appropriate to define a microreactor/microchannel as having, at its narrowest part, a cross-section in a plane perpendicular to the flow direction which is sized so that the largest circle which can be drawn in the cross-section has a diameter of less than imm (and preferably less than 250 μm). In other words, if the cross-section is such that a circle with a diameter of greater than imm can be drawn within the cross-section, it will not operate as a microchannel.

In principle, there is no limit to the reduction of the volume of the system, since laminar flow conditions will continue to prevail as the volume is reduced.

The apparatus may comprise a multi-channel device. As explained above, this means that the reagents flow along the channels/microchannels and react when combined at flow junctions. The number of channels will depend upon the experiment, although typically 4 channels are employed, with a separate reagent being injected into each channel, as demonstrated in FIG. 1. The microchannels may be formed in a microfluidic chip. The chips may be made of glass, quartz or plastic.

The apparatus may cause the fluids to move by pressure-driven flow, electrokinetic flow, or a combination of the two. The apparatus may include a pump and, in particular, a nano-flow pump. A nanoflow pump may be defined as a pump that pumps fluids at less than 1 mL per minute. Within the embodiment of the subject matter disclosed herein the nanoflow pump may operate up to 2 mL per minute. The purpose of this is to drive the reagents along the channels. This may be a multi-channel nano-flow pump if a multi-channel device is used. Typically a 4-channel nano-flow pump is used if there are 4 channels. The use of such nanoflow pumps has previously been restricted to two-dimensional liquid chromatography. Nanoflow pumps have never previously been used in assays similar to those of the subject matter disclosed herein. A servo motor may be used to drive the pump.

In one embodiment, the pressure applied by the pump to drive the liquid is controlled via a feedback system in which the flow-rate is measured downstream of the pump and used to regulate the pressure applied by the pump. This feedback control enables the pressure applied by the pump to respond promptly to sudden but short-lived (defined as less than 5 seconds, preferably less than 3 seconds and more preferably less than 1 second) effects that cause sudden changes in flow, such as transient blockages. Thus, the flow-rate does not differ from the intended flow rate for a significant length of time. The flow-rate may be measured at intervals or, preferably, continuously. It may be measured at intervals of from 1 μs to 1 s, preferably 1 ms to 1 s and more preferably from 1 ms to 100 ms. This facilitates a “real” measure of the concentration of components in the assay and not an assumed concentration that is based on assumed flow calculated indirectly from the pressure applied to the pumping system.

In a further embodiment, the pressure may be increased or decreased quickly to provide rapid changes in volumetric flow-rate. Thus, in one embodiment, the time over which the concentration varies (i.e. the length of the step(s)) may be 20 seconds or longer.

The apparatus may comprise a degasser. Alternatively, a degasser may not be used.

The system may further have a transfer mechanism to transfer reagents from an array of reagent reservoirs to the channel structure. The operation of the transfer mechanism may be controlled by a computer. If chips are used, reagent reservoirs may be wells on the chip or capillaries attached to the chip. Thus, reagents may be introduced either from wells on the chip or from capillaries attached to the chip. An autosampler may be used to introduce the reagent or compound into the system. Steel valves may be used to introduce the reagent or compound into the system. These steel valves may be nano-volume steel valves.

The apparatus may comprise an x,y,z-positioning stage, the function of which is to provide correct positioning of the point of detection (typically the point of detection of fluorescence), to the centre of the microfluidic flow.

An incubator may be used to house the microchannel device so that the reaction between the compound and the target occurs at a stable temperature. Typically the incubator is maintained at physiological temperature for biochemical reactions.

The components of the apparatus may be interconnected with capillaries. The capillaries may be silica capillaries. Preferably they are pre-cut and polished fused silica capillaries. The internal diameter of the capillaries may be of any dimension. Preferably, the capillaries are of between 10 μm and 50 μm internal diameter and 325 μm and 425 μm outer diameter. Preferably, the capillaries are of 30 μm internal diameter and 375 μm outer diameter. Each valve may also have a capillary loop acting as a reagent reservoir. The use of nano-bore capillaries and nano-volume valves enables low dead volumes and fast transit times to the microchannel device.

As described above, when the method of the subject matter disclosed herein involves more than one step of adding the compound to a flow of the target, the relative change (as defined above) in concentration in each step may be the same. In one embodiment, the relative change in a step is between 1-fold and 200-fold. Preferably it is between 5-fold and 120-fold. More preferably it is between 10-fold and 80-fold. Even more preferably it is between 20-fold and 60-fold. Most preferably it is 40-fold.

Each step of the subject matter disclosed herein may independently be performed for any length of time. Each step of the subject matter disclosed herein is performed, in rising degrees of preference, for greater than one second, for from 1 second to 10 hours, for from 10 seconds to 1 hour, for from 30 seconds to 45 minutes, for from 30 seconds to 30 minutes, for from 30 seconds to 20 minutes, for from 30 seconds to 10 minutes, for from 30 seconds to 4 minutes or, most preferably for 1 minute.

In one embodiment of the subject matter disclosed herein, there is a linear 40-fold decrease in concentration over two minutes in each step.

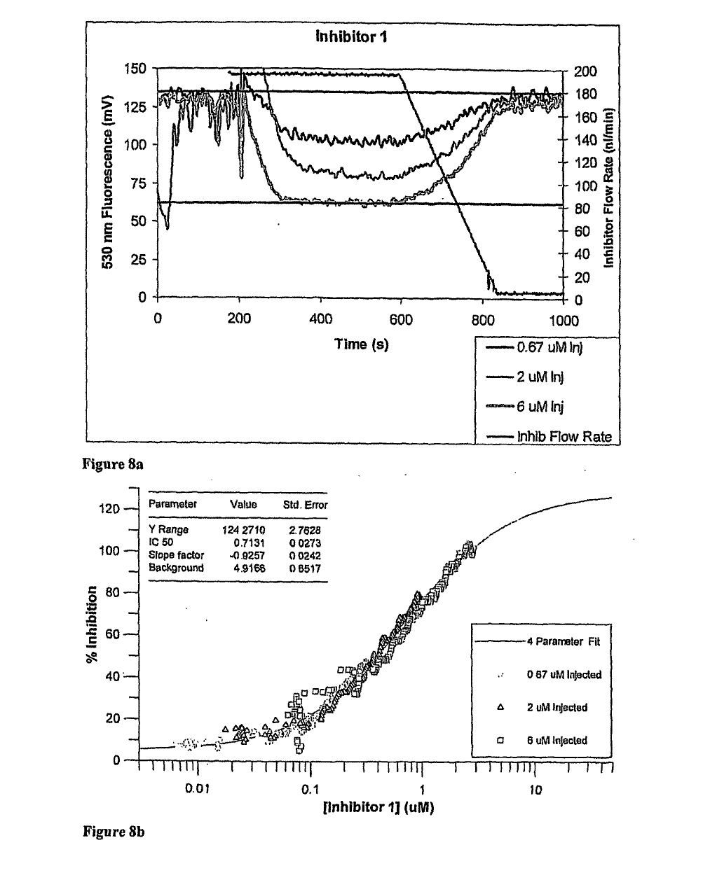

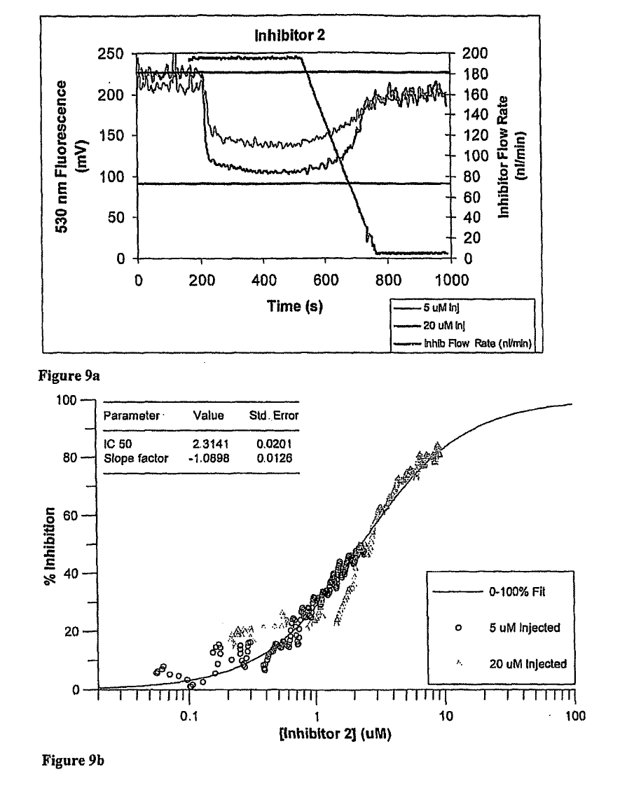

The data are most typically fitted to a four parameter logistic model but may be fitted to other models that can be used to characterise inhibition/activation of targets. Indeed, a model with any number of parameters may be used. The number of parameters may be five, four, three or two. The two-fold symmetry of the four and three parameter logistic models (see FIGS. 8 and 9) means that, provided data of sufficient quality are collected, the maxima and minima do not need to be derived experimentally for individual inhibitors/activators.

The data derived from each step may be combined to produce one overall set of data spanning the entire array of concentrations tested. These data can be used to produce one overall description of best fit to the preferred mathematical model. The presence of overlap between the ranges assists the process of overlaying the data and acts to provide reference areas or correction factors which facilitate the fitting of the total data from all steps to the preferred model. Accuracy in the determination of values, such as ICₓ and ECₓ values, is thereby improved. Furthermore, the large amount of data and the fact they span a large array of concentrations allows an improved mathematical description of the mode of action of the compound with the target. It may even facilitate detailed mechanistic studies to be carried out simultaneously with an experiment to determine values, such as ICₓ and ECₓ values.

As described above, where the method involves more than one step comprising the addition of the compound to a flow of the target, certain steps may only be performed when, within a certain other step or certain other steps, the compound yields a result, or combination of results, that falls within a particular range. Preferably, certain steps may only be performed when, within a certain other step or certain other steps, the compound yields an ICₓ/ECₓ value, and typically an IC₅₀/EC₅₀ value, or a combination of the ICₓ/ECₓ, and typically IC₅₀/EC₅₀, values determined in each step, that falls within a particular range. In particular, all the steps other than the first step may only be performed when, within the first step, the compound yields an ICₓ/ECₓ, and typically an IC₅₀/EC₅₀, value that falls within a particular range. In a modification of this, a step may only be performed when, within the previous step, the compound yields an ICₓ/ECₓ, and typically an IC₅₀/EC₅₀, value that falls within a particular range. This approach eliminates unnecessary testing of inactive compounds or weakly active inhibitors/activators, reduces wastage of the compound or other reagents and generally rationalises the efficiency of the early stages of drug development.

When the method of the subject matter disclosed herein is used to probe a range of compound concentrations that provides a dilution factor similar to that assayed in a microtitre plate assay (e.g. biochemical research commonly involves a 59000-fold dilution, resulting from a 3-fold serial dilution made up of 11 compound concentrations), a number of steps may be performed. For example, three 40-fold concentration gradients could be used to give a 48000-fold dilution (including a small overlap in the gradients).

As described above, a decision-making process may be used to determine whether the compound should be subjected to further concentration gradients. The decision-making process may be manual or automated. Typically, the decision will depend on whether the value, such as an ICₓ or ECₓ value, for a particular concentration gradient falls within a predetermined range. In one embodiment, inhibitors/compounds that are subjected to a first concentration gradient and found to have IC₅₀ values greater than 10 μM are not subjected to further concentration gradients because such weak/inactive inhibitors are generally not desirable in drug discovery.

The subject matter disclosed herein also relates to a method of performing biological assays in which the experiments are performed at a temperature other than physiological temperature. Typically biological experiments are performed at physiological temperature so that the observed biochemistry conforms to the biochemistry that would take place in vivo. However, this means that reactions often occur at a rate lower than if they were performed at a higher temperature. It has been discovered that the efficiency of in vitro experiments can be increased by increasing the temperature. Conversely, it may sometimes be useful to perform the reactions at a temperature below physiological temperature, e.g. if one wants to slow a reaction down in order to analyse a reaction that would otherwise be very fast.

Although reactions performed at increased temperature may yield results that differ from those in physiological conditions, their results may still be of use. For example, if a series of inhibitors/activators/compounds were assayed using the aforementioned methodology, but at a temperature greater than physiological temperature, then the ICₓ/ECₓ values would differ from their physiological values. However, if the inhibitors/activators were ranked in order of the ICₓ/ECₓ values determined in said experiment, then the order would be the same as if the experiments were performed at physiological temperature. Therefore, performing the assays at increased temperatures provides a useful way of determining which, out of a number of inhibitors/activators/compounds, are the least/most potent. The advantage of this method is that experimental speed is increased.

In a preferred embodiment, the concentration range that contains the highest concentration (concentration range) is performed first. All compounds are assayed over this concentration range and, if their value (as defined elsewhere) does not fall within the predetermined range, they are not assayed at other concentration ranges. Conversely, if their value does fall within the predetermined range, they are assayed at the concentration range (concentration range 2) that has a highest concentration higher than the highest concentration of the other ranges (apart from concentration range 1). The same decision is made according to the value determined in concentration range 2 and the process is repeated in subsequent concentration ranges, if they are performed. This approach is herein termed the ‘triage’ process.

The triage assay process eliminates unnecessary testing of compounds which do not satisfy the activity criteria and thus reduces wastage of the compound and other reagents and generally rationalises the efficiency of the early stages of drug development.

Most importantly, the triage assay process allows the determination of values (e.g. ICₓ or ECₓ values) even when they are unknown.

The aforementioned decision-making process may be performed by a computer, preferably a computer that has been pre-programmed to reject compounds with values (e.g. ICₓ or ECₓ values) above/below a certain value. The computer may be of any type. Preferably, it uses a WINDOWS® or a MAC® operating system.

The decision-making process may use an algorithm, such as a Simplex algorithm or a genetic algorithm or a combination thereof. Instead of, or as well as, an algorithm, a neural network could be used. Such algorithms can be used to decide, without direct user input, both whether a subsequent step is to be performed and, if so, which concentration range and gradient is to be used.

The skilled person will appreciate that any general reference herein to “value” means “a value that allows the effect that a compound has on a target to be compared with the effect that another compound has on the target” and thereby incorporates ICₓ/ECₓ values. The skilled person will also appreciate that some instances of “ICₓ/ECₓ values” and related terms used herein may be generalised to refer to “values”. As an example, the subject matter disclosed herein may be used to calculate other parameters such as Kᵢ and K_{d} values and measures of toxicity in addition to ICₓ/ECₓ values.

Typically, the activity of the target will be assessed by reference to its interaction with a substrate or a ligand.

As used herein, a “substrate” is an entity that undergoes reaction with the target. If the target is an enzyme, this reaction may cause the substrate to break up into products or may involve the formation of a new entity from two or more substrate moieties.

As used herein, a “ligand” is an entity that binds to the target to some extent but does not necessarily react with the target to form a new chemical entity or entities that is/are derived from the ligand.

The skilled person will be aware of suitable techniques for assessing target activity that may be used in conjunction with the subject matter disclosed herein. As mentioned above, the target activity may be monitored directly or indirectly. Direct monitoring of the target activity may be defined as any case where some property of the target itself is monitored. Indirect monitoring of the target activity may be defined as any case where some property of a species other than the target itself is monitored. Indirect monitoring includes the situation where the target or the substrate undergoes a further reaction and this reaction is monitored in some way. Indirect monitoring also includes the situation where an analysis of the concentration of the products of the reaction in relation to the concentration of substrate before it underwent reaction with the target is performed.

Any suitable substrate may be involved in the method. Any suitable ligand may be involved in the method. The method may involve both a substrate and a ligand. The substrate and/or ligand may be labelled with a moiety that is fluorescent or luminescent. This moiety may be fluorescent or luminescent either directly or indirectly. Labelling with a moiety that is fluorescent or luminescent may be of use if, say, fluorescence intensity, fluorescence lifetime, fluorescence polarization or luminescence techniques are employed to determine the displacement of labelled ligand/substrate by the compound.

Targets involved in the present method may be made by recombinant DNA technology, for instance by expressing a gene for the protein in a suitable host cell. Suitable techniques forming the state of the art may be used. These include the techniques discussed in References 11 and 12.

The target and/or the substrate/ligand may be prepared in any buffer understood by those skilled in the art to be suitable. Suitable buffers include:

100 mM HEPES (N-(2-Hydroxyethyl)piperazine-N'-(2-ethanesulfonic acid)) (pH 7.4), 300 mM NaCl, 20 mM CaCl₂, 2 μM zinc acetate, 1.2 mM CHAPS (3-[(3-cholamidopropyl)simethylammonio]-1-propanesulfonate), 0.04% (w/v) sodium azide in MilliQ purified water;

125 mM HEPES (pH 7.5), 25 mM MgCl₂, 2.5 mM CHAPS, 0.04% (w/v) sodium azide, 2 mM dithiothreitol (DTT) in MilliQ purified water; or

100 mM HEPES (pH 7.5), 20 mM MgCl₂, 2 mM CHAPS, 0.04% (w/v) sodium azide and 2 mM DTT (added just prior to use) in MilliQ purified water.

The target may be unstable and therefore may require introduction into the apparatus as a complex (see example 2 below).

The compound being tested may be of any type and may be prepared by any method known in the art. Preferably it is an inhibitor/activator. The compound may be a small molecule with a molecular weight less than or equal to 500 Da. The compound may be a larger complex, such as an antibody. The compound may be a drug candidate or a new chemical entity. It may be stored in any solvent. Preferably, the compound is soluble in said solvent. In one embodiment, the compound is water-soluble. If the compound is an inhibitor/activator, it may be reversible or irreversible. If the compound is an inhibitor/activator, it may be competitive, non-competitive, uncompetitive or mixed.

In one embodiment of the subject matter disclosed herein, H⁺, and optionally any isotopes thereof, are excluded from the definition of compound and, preferably, the definition of inhibitor/activator. In another embodiment of the subject matter disclosed herein, the compound, and preferably the inhibitor/activator, has a molecular weight of greater than 3 Da, more preferably greater than 5 Da, yet more preferably greater than 15 Da and most preferably greater than 30 Da.

The compound may be added in neat form but is more typically added in a suitable vehicle, e.g. in a mixture or solution that is similar to the mixture or solution in which the target is present and/or assayed. Alternatively, the compound may comprise precursors of the compound which are in equilibrium with the compound itself or precursors of the compound which form the compound after they have come into contact with the flow of the target or some other reagent.

Those skilled in the art will appreciate that elements of the apparatus may be cleaned using appropriate solvents at any stage and, in particular, after each step. Any of 2% (w/v) lithium dodecyl sulfate (LDS), water, acetone and methanol may be used to this end.

The method may involve any suitable vehicle for the reaction. This may be any suitable aqueous assay buffer. In one embodiment a methanol in water mobile phase is used. This methanol in water mobile phase may be 2% (v/v).

The method may be performed at any temperature. The target, substrate/ligand and compound may be stored at specified temperatures prior to the assay. The storage at a specified temperature prior to the assay may occur in an autosampler, if one is used.

Any total flow rate through the apparatus may be used. Preferably, this remains the same throughout the experiment. The total flow rate used will depend on the apparatus employed. The total flow rate may be, in rising order of preference, between 10 nl/min and 5 pl/min, between 100 nl/min and 1 μl/min, between 200 nl/min and 600 nl/min and between 300 nl/min and 500 nl/min.

If a multi-channel device is used, the substrate/ligand may be injected into one channel and the target into another channel. Each channel may flow at any rate. Alternatively, the substrate may be injected into the same channel as the target or the compound.

When a sample is introduced into the flow channels, it commonly has a “sample front” that will undergo Taylor dispersion. This dispersion means that the concentration of the sample across the sample front varies. For accurate results, it is necessary to ensure that the sample front is well past the point of detection and that the concentration of compound analysed is equivalent to that calculated from the flow rate and concentration of the sample introduced into the system. Therefore, prior to the injection of the compound, the flow rate in the substrate/ligand and target channels may be temporarily increased in order to rapidly equilibrate concentrations of the target and substrate/ligand at the detection point. This reduces experimental time and increases throughput. A flow rate of 500 nl/min may be used for this purpose.

The compound may be injected into another channel. Preferably, this is done when a stable target-substrate/ligand signal is achieved. Preferably, the highest concentration of compound is first injected into this other channel. The flow rate in this other channel may be increased in order to equilibrate concentrations at the point of detection. A flow rate of 500 nl/min may be used for this purpose. The flow rate of the compound may then be reduced prior to the step of continuously varying the concentration of the compound with time.

The continuous concentration gradient may be applied by varying the flow rate of the compound. The continuous concentration gradient may be of any nature. A 40-fold decrease in concentration may, for example, be used (e.g. a decrease in flow rate from 195 nl/min to 5 nl/min).

Preferably, a suitable vehicle is added in another channel in order to maintain a constant total flow rate whilst the flow rate of the compound is varied.

The compound may then be flushed out of the system using the reaction vehicle.

If there is more than one step of continuously varying the concentration of the compound with time, the procedure for the other steps is as described above. As discussed above, the range of concentrations used and the nature of the concentration gradient in subsequent steps may differ from the first step.

In an embodiment of the subject matter disclosed herein, the compound used in the first concentration gradient step may be diluted and the diluted compound used in the second concentration gradient step. In a further embodiment, this diluted compound is further diluted and then the further diluted compound is used in a third concentration gradient step. Further embodiments of the subject matter disclosed herein contain further dilution and concentration gradient steps.

The subject matter disclosed herein may be used in conjunction with a variety of detection methods, including techniques relating to fluorescence intensity (FI), time-resolved fluorescence (TRF), fluorescence lifetime (FL), fluorescence polarization (FP) (see below for technical details), luminescence, Raman spectroscopy, mass spectrometry and electrophoresis. These detection methods may be used to determine the target activity on the basis of enzyme activity or ligand binding. FI, TRF and FP may be used to measure the concentration of a fluorophore product of an enzyme reaction. FL or FP may be used to determine the displacement of fluorescently-labelled ligands by the compound.

The skilled person will appreciate that the precise mechanism in some cases may differ. For example, it may actually be a derivative of the compound that displaces the fluorescently-labelled ligands.

An FI measurement system involves excitation of a fluorophore by a laser. This may be a diode pumped solid state laser. Any excitation wavelength may, in theory, be used although the excitation wavelength chosen will depend on the fluorophore. An excitation wavelength of 532 nm may be used when the fluorophore is Cy3B, for instance.

Detection may be by a confocal optical head. Detection may occur at any emission wavelength and, again, the emission wavelength will depend on the fluorophore. An emission wavelength of 560 nm may be used when the fluorophore is Cy3B. The detector may comprise a photomultiplier tube (PMT). The data may be acquired from the PMT by any suitable means. In the case of an analogue PMT, the data are acquired using an analogue data acquisition card such as the PCI-6052E card [National Instruments] controlled by suitable software. Any number of data samples per second may be used. Preferably, this number varies between an average of 200 and 2000 samples per second. Preferably an average rate of 1000 samples per second is used.

At least one fluorometric detector may be used. At least one backscatter detector may also be used. In one embodiment, where the technique is a multicolour and multifluorophore one, at least two fluorometric detectors may be used in conjunction with a backscatter detector to facilitate the measurement of at least two fluorophores with distinct spectral characteristics.

The laser and the PMT may be coupled to the optical head using optical fibres.

A fluorescence resonance energy transfer (FRET) assay provides an example of a way in which the subject matter disclosed herein may be used in conjunction with an FI technique. FRET is suitable for, for example, inhibition studies of proteases. It may, for example, be used for matrix metalloproteinase 12 (MMP12) studies.

When FP is measured, the apparatus may be the same as that described for the FI system, except that an FP measurement system is used. Any excitation wavelength of linearly polarised light may be used. A laser excitation wavelength of 532 nm or 488 nm may be used if the fluorophore is Cy3B or fluorescein, respectively. Detection occurs in planes both parallel and perpendicular to the plane of the incident excitation light. Detection may occur at any emission wavelength, which is dependent on the fluorophore used. Detection may occur at an emission wavelength of 570 or 532 nm if the fluorophore is Cy3B or fluorescein, respectively. Two single photon counting modules (SPCMs) may be used for the detection, one for each of the parallel and perpendicular channels. The FP data may be acquired from the SPCMs using a digital counter card.

When FL is measured, the apparatus may be the same as that described for the FI system, except that a lifetime measurement system is used whereby time-resolved fluorescence intensity maybe derived alone or used to derive FL. An example of a lifetime measurement system is the TimeHarp 200 with accompanying pulsed laser and synchronisation electronics (PicoQuant GmbH). Any excitation wavelength may be used. Preferably, a laser excitation wavelength of 488 nm, 532 nm or 635 nm is used in conjunction with the fluorophores fluorescein, Cy3B and Cy5, respectively. Detection may occur at any emission wavelength but is preferably 530 nm, 570 nm or 670 nm when using fluorescein, Cy3B and Cy5, respectively. A PMT may be used for time-correlated detection. If a PMT is used for detection, lifetime data may be acquired from the PMT using a time-correlated photon counting card, such as the TimeHarp 200 (PicoQuant GmbH), controlled by suitable software.

Any suitable software may be used to perform background correction, determination of reagent concentrations, calculation of % inhibition or % activation, calculation of % ligand binding, determination of ICₓ/ECₓ, determination of Kᵢ, determination of K_{A} and determination of K_{d}. In one embodiment, software written using Labview Express 7 [National Instruments Co.] is used in conjunction with appropriate instrument driver software (i.e. *.dll's—PicoQuant GmbH).

Data from the detection methods may be analysed using a variety of methods.

If fluorescence/luminescence data are collected, these should be corrected for background fluorescence/luminescence (i.e. the fluorescence/luminescence resulting from the presence of any target and buffer reagents. Preferably, the background signal is determined by providing samples of substrate/ligand at different concentrations and adding a fixed amount of target to each one of these samples. The concentration of substrate or ligand (determined using flow data) is then plotted on the x-axis against fluorescence/luminescence on the y-axis and then a linear regression is performed to determine the best-fit line. Background fluorescence/luminescence is determined as the y-intercept, i.e. extrapolating the value of fluorescence/luminescence for a substrate or ligand concentration of 0 nM.

The concentration of compound may be ascertained by considering the flow rates. Specifically, the concentration of the compound ([cmpd]) is calculated according to the following equation:

$$[ cmpd ] = injected [ cmpd ] \times \frac{flow rate of compound}{total flow rate} .$$

The fluorescence/luminescence data at various flow rates of compound may be obtained, thereby providing fluorescence/luminescence data at various compound concentrations.

The signal window may be determined by calculating the difference between the fluorescence/luminescence for the full reaction (target with substrate or ligand) and the background fluorescence/luminescence.

The percentage inhibition/activation may be calculated thus:

$$\% inhibition = \frac{( \begin{matrix}signal in presence of compound - \\ background signal\end{matrix} )}{( \begin{matrix}signal from full reaction - \\ background signal\end{matrix} )} \times 100 \% .$$

An ICₓ/ECₓ value may be determined by plotting concentration of compound on the x-axis against percentage inhibition on the y-axis and fitting a suitable curve. The curve may be fitted using any suitable mathematical technique. Preferably, it is fitted using a four parameter logistic model. The equation of a four parameter logistic model (to be used in conjunction with the determination of an IC₅₀/EC₅₀ value) is as follows:

$$a + \frac{( b - a )}{( 1 + {( \frac{x}{c} )}^{d} )}$$

where a is the background or lowest signal, b the highest signal, c is the IC₅₀ and d is the slope.

As a measure of assay performance, a Z′ factor may be calculated thus:

$${Z}^{′} factor = 1 - \frac{[ \begin{matrix}( 3 \times SD of High Control ) + \\ ( 3 \times SD of Low Control )\end{matrix} ]}{Mean High Control - Mean Low Control}$$

where:

SD=standard deviation;

High Control=signal resulting from target and substrate or ligand, and

Low Control=background signal.

Other aspects and features of the subject matter disclosed herein are set forth in the description of exemplary embodiments which now follows. The subject matter disclosed herein is not limited to the examples described below, but may take on many other guises, forms and modifications within the scope of the claims.

Embodiments of the subject matter disclosed herein are shown in the accompanying figures in which:

FIG. 1 shows a schematic diagram of the injection of an inhibitor, buffer, target (enzyme) and substrate into an apparatus at different flow rates to give a total flow rate of 400 nL/min. Two possible points of monitoring the activity of the target are shown (primary detection, which might typically comprise direct monitoring of the activity of the target, and secondary detection, which might typically comprise indirect monitoring of the activity of the target).

FIG. 2 provides an illustration of how the components of an apparatus may be linked in one embodiment of the subject matter disclosed herein. It also shows how the components are controlled and the data are processed. Four fluids are pumped, preferably using a nanoflow pumping system (P1-4). Four pump fluid lines are connected to four valves (V1-4), which facilitate the introduction of biological reagents into the four pump fluid lines and thence to a microfluidic microchip (MFC). The reagents are introduced into the valves using an autosampler (AS). The detection system (DS) is located close to MFC to enable changes within the microchannels of MFC to be measured. The whole system is controlled externally via a computer (PCA).

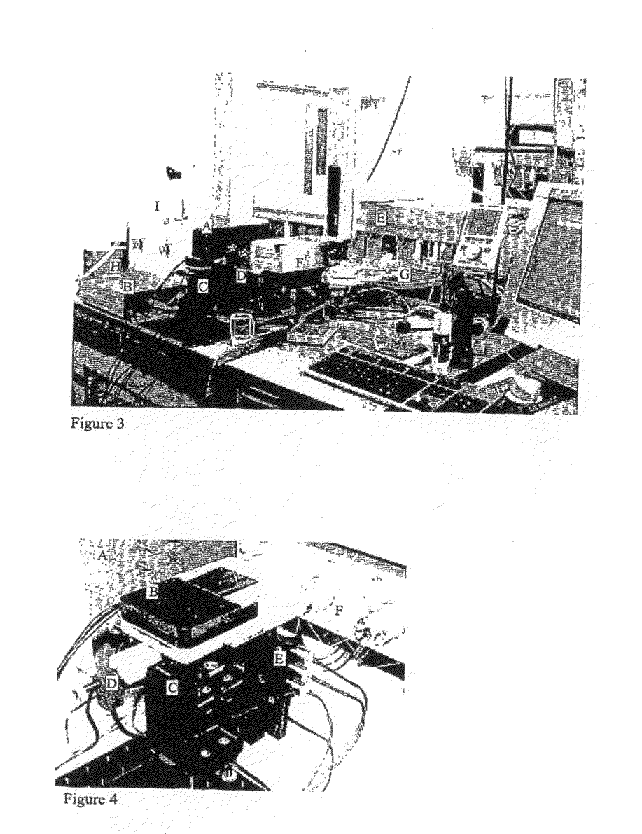

FIG. 3 provides a photograph of a fluorescence intensity microbiochemistry platform suitable for performing the methods of the subject matter disclosed herein. The reference signs in FIG. 3 correspond to the following elements: A=pump; B=laser; C=x,y,z-stage positioner & incubator housing containing microchannel device; D=four injection nano-volume valves; E=autosampler; F=4° C. reagent vial storage; G=room temperature reagent reservoirs; H=degasser for mobile phase; and I=2% (v/v) methanol in water mobile phase reservoir.

FIG. 4 provides a photograph of an incubator housing and an x, y, z positioning stage, suitable for use in conjunction with the subject matter disclosed herein. The reference signs in FIG. 4 correspond to the following elements: A=pump; B=incubator housing for microchannel device; C=x,y,z-positioning stage; D=one of three piezo stage controllers; E=optical head with fibre optic connections; F=four injection valves.

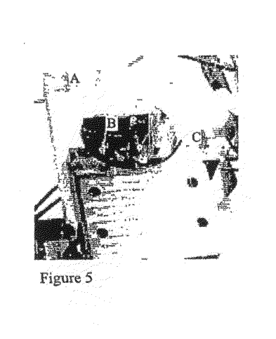

FIG. 5 provides a photograph of a microchannel device in an incubator, suitable for use in conjunction with the subject matter disclosed herein. The reference signs in FIG. 5 correspond to the following elements: A=incubator housing; B=microchannel device showing capillary connections; C=optical head.

FIG. 6 illustrates an embodiment of the subject matter disclosed herein in which overlapping concentration gradients are used to calculate the IC₅₀ value of an inhibitor. The top graph of FIG. 6 shows the use of three steps to perform three different concentration gradients of 20 μM to 500 nM (concentration gradient A), then 571 nM to 14 nM (concentration gradient B) and then 16 nM to 410 μM (concentration gradient C). In this example, inhibitors that produce <20% inhibition in the first gradient are not subjected to the second and third gradients. Likewise inhibitors that produce <20% inhibition in the second gradient are not subjected to the third gradient. For a particular target (in this case a protease called caspase), the bottom graph shows the number of inhibitors that would have to be assayed in each concentration gradient. This bottom graph is calculated based on data collected for 2205 structurally dissimilar inhibitors using conventional microtitre plate technology. It is therefore clear that the ‘triage’ approach eliminates unnecessary testing of inactive compounds or weakly active inhibitors/activators and therefore reduces reagent wastage and increases speed. In this example only 90 inhibitors are assayed in the third concentration gradient. Furthermore, the extended concentration range that may be probed means that sets of inhibitors/activators/compounds whose values (e.g. ICₓ/ECₓ values) are unknown can be tested.

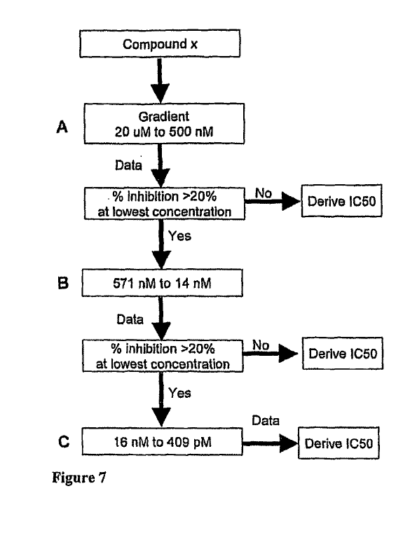

FIG. 7 provides an example of a decision-making process that could be used in the ‘triage’ approach. In this example, the method is to determine IC₅₀ values and the assay corresponds to that shown in FIG. 6. Letters A, B and C refer to the three concentration gradients mentioned in FIG. 6. If an inhibitor is subjected to concentration gradient A, concentration gradients A and B or concentration gradients A, B and C, the IC₅₀ is calculated from data collected from one, both or all three concentration gradients, respectively. In these examples, inhibitors/compounds that produced <20% inhibition in one concentration gradient were not subjected to further concentration gradients. For a given assay, the user may choose any percentage inhibition/activation threshold(s) that a compound must fall within during a particular concentration gradient to be excluded from further concentration gradient(s). Furthermore, for a given assay, the user may determine the extent to which and manner in which the concentration gradients overlap.

FIGS. 8a and 8b illustrate data collected using an embodiment of the subject matter disclosed herein in which adjacent concentration gradients overlap to some extent. In this example, the target was the proteinase MMP12 and the inhibitor was an inhibitor of MMP12. Fluorescence intensity (FIG. 8a) was used to monitor the MMP12 activity. The inhibitor concentration was calculated from flow-rate data provided by the pump. The MMP12 activity was expressed as a percentage of MMP12 activity in the absence of an inhibitor. The three overlapping gradients used had, respectively, highest concentrations of approximately 3, 1 and 0.33 μM and each gradient had a lowest concentration 40-fold lower than the highest concentration for that gradient. The fluorescence intensity data were converted into % inhibition data and the data were fitted to a four parameter logistic model, which was then used to determine the IC₅₀ (FIG. 8b).

FIGS. 9a and 9b also illustrate data collected using the overlapping gradient approach of the subject matter disclosed herein. Again, the target was MMP12 and fluorescence intensity (FIG. 9a) was used to monitor the protease activity. The inhibitor concentration was calculated from flow-rate data provided by the pump. The MMP12 activity was expressed as a percentage of MMP12 activity in the absence of an inhibitor. Two horizontal bars are visible in FIG. 9a; the top bar represents 100% MMP12 activity and the bottom bar represents 0% MMP12 activity. Two overlapping gradients were used, with respective highest concentrations of approximately 10 and 2.5 μM. Each gradient had a lowest concentration 40-fold lower than the highest concentration for that gradient. The fluorescence intensity data were converted into % inhibition data and the data were fitted to a three parameter logistic model, which was then used to determine the IC₅₀ (FIG. 9b).

### Examples

A system for measuring fluorescence intensity (FI) may be used in conjunction with the subject matter disclosed herein. In particular, the following experimental set-up has been used.

A microbiochemistry FI assay platform (see FIGS. 1 to 3) was used for continuous detection. A flowing reagent system, comprising the following components, was used: a degasser [SDU-2006, ProLab] for a 2% (v/v) methanol in water mobile phase flowing through the system; a four-channel nano-flow pump [Eksigent Technologies] independently flowing reagent or compound at between 5 and 500 nl/min per channel; an autosampler to introduce reagent or compound [HTS PAL with Cycle Composer software, CTC Analytics AG] into the system via four nano-volume steel valves [C2N-4306D, Valco Instruments Co. Inc]; x,y,z-positioning stage and motors [Bookham New Focus] to locate the point of detection at the centre of a microfluidic channel, which was within a glass microchannel device [Micronit Microfluidics BV]; an incubator to house the microchannel device [Linkam Scientific Instruments Ltd] which was maintained at 37° C. using a temperature controller [INC37, Linkam Scientific Instruments Ltd]; connections between the pump/microchannel device and the valves are pre-cut and polished fused silica capillaries [Polymicro Technologies Inc.] of 30 μm internal diameter and 375 μm outer diameter, and each valve also has a capillary loop acting as a reagent reservoir. The use of micro-bore capillaries and nano-volume valves enables low dead volumes and fast transit times from the valves to the microchannel device. Capillaries were attached to the microfluidic chip using NANOPORT™ [Upchurch Scientific Inc.] connector ferrules [N-123-04] and nuts [catalogue number F-123H], in conjunction with a polymeric [acetal copolymer] microchannel chip holder (as illustrated in FIG. 5). The FI measurement system [Genapta Ltd, WO 03/048744 A2] involves excitation of fluorophore by a diode-pumped solid state laser with an excitation wavelength of 532 nm. Detection was by a confocal optical head at an emission wavelength of 570 nm with an analogue photomultiplier tube (PMT). The laser and the PMT were coupled to the optical head using optical fibres. The fluorescence intensity data were acquired from the PMT using an analogue PCI-6052E card [National Instruments Co.] controlled by software written using LabView 7 Express [National Instruments Co.] with averaging of 1000 samples per second. The background correction, reagent concentration determination, % inhibition calculation and IC₅₀ value determination were performed using software written using Labview Express 7 [National Instruments Co.].

A system for measuring fluorescence polarization (FP) may be used in conjunction with the subject matter disclosed herein. In particular, the following experimental set-up has been used.

The apparatus was the same as that used in the fluorescence intensity system, except that the following components were used: a 4-channel nano-flow pump [Eksigent Technologies]; an x,y,z-positioning stage with motors [Physik Intrumente (PI) GmbH & Co KG] and controlling software [Genapta Ltd]; an FP measurement system [Genapta Ltd] employing a laser excitation wavelength of 488 nm with detection at emission wavelengths of 530±15 nm, in planes that are both parallel and perpendicular to the plane of the incident light, with two single photon counting modules (SPCMs [SPCM-AQR, Perkin Elmer], one for each of the parallel and perpendicular channels). The FP data was acquired from the SPCMs using a digital card and software [Genapta Ltd].

A system with a 532 nm laser excitation wavelength and detection at an emission wavelength of 570 nm [Genapta Ltd] has also been used.

A system for measuring fluorescence lifetime (FL) may be used in conjunction with the subject matter disclosed herein. In particular, the following experimental set-up has been used.

The apparatus was the same as that used in the fluorescence intensity system, except that the following components were used: a degasser [DG-2080-53, Jasco]; an x,y,z-positioning stage and motors [Physik Intrumente (PI) GmbH & Co KG]; a lifetime measurement system [PicoQuant GmbH]: a laser excitation wavelength of 635 nm with detection at an emission wavelength of 670 nm with a PMT. Lifetime data was acquired from the PMT using a photon counting card and software [PicoQuant GmbH].

Systems with 488 nm and 530 nm excitation wavelengths and detection wavelengths at 530 nm and 570 nm, respectively, have also been used.

### Example 1

The FI system has been successfully used in accordance with the subject matter disclosed herein to perform an assay for inhibitors of matrix metalloproteinase 12 (MMP12).

Specifically, a fluorescence resonance energy transfer (FRET) assay for MMP12 inhibitors was used. MMP12 cleaves a substrate peptide, labelled with both a carboxyfluorescein (FAM) donor fluorophore and a tetramethyirhodamine (TAMRA) acceptor fluorophore, liberating the donor fluorophore with a resulting increase in fluorescence. The assay involved human, recombinant MMP12 catalytic domain (residues G106-N268) expressed in E coli and FAM-TAMRA labelled substrate peptide [fam-Gly-Pro-Leu-Gly-Leu-Phe-Ala-Arg-Lys-TAMRA-NH2 synthesised in-house]. The substrate and enzyme were prepared to the required concentrations in assay buffer: 50 mM HEPES (N-(2-hydroxyethyl)piperazine-N′-(2-ethanesulfonic acid)) (pH 7.4), 150 mM NaCl, 10 mM CaCl₂, 1 μM zinc acetate, 0.2% (v/v) Tween 80 (polyethylenesorbitan monooleate), 0.02% (w/v) sodium azide in MilliQ purified water [all buffer reagents were from Sigma, except HEPES which was from Invitrogen]. 2% (w/v) lithium dodecyl sulfate (LDS) [from Sigma] was used to clean the injection syringe after substrate and enzyme injection. 2% (w/v) LDS was also used to clean the microchannel device as required. Inhibition of MMP12 was demonstrated using two small molecule inhibitors, known to have an inhibition constant (Kᵢ) of approximately 290 nM (Inhibitor 1) and approximately 1 mM (Inhibitor 2) from a microplate-based MMP12 assay. Each inhibitor was diluted with assay buffer from a 10 mM stock, prepared in neat dimethylsulfoxide (DMSO), to the required concentration.

Initially the pump continuously flowed 2% (v/v) methanol in water, which constituted the mobile phase for the assay system, through all four channels. The reagents and inhibitors were then introduced into the system, replacing the mobile phase. The total flow rate in the system was maintained at 400 nl/min. The reaction was performed at 37° C. Prior to injection, the enzyme, substrate and inhibitor were stored at 4° C. in glass vials in a cooled tray on the CTC Analytics HTS Pal autosampler. 400 nM substrate peptide was injected into one channel flowing at 100 nl/min. The injection syringe was then cleaned in 2% (w/v) LDS, stored in a room temperature CTC reagent reservoir, followed by 100% (v/v) methanol and finally water. 19 nM MMP12 enzyme was injected into a second channel flowing at 100 nl/min and the syringe needle was cleaned as above. The remaining two channels flowed with assay buffer at 100 nl/min per channel. The flow rate was increased in the substrate and enzyme channels to 500 nl/min for 3 minutes to quickly equilibrate concentrations at the detection point. The final concentrations in the assay were: 1 μM substrate peptide, 1 nM MMP-12, 50 mM HEPES (pH 7.4), 150 mM NaCl, 10 mM CaCl₂, 1 μM zinc acetate, 0.02% Tween 80, 0.02% (w/v) sodium azide. Once a stable enzyme-substrate (ES) signal was achieved, the highest concentration of inhibitor (for inhibitor 1=6 μM; for inhibitor 2=20 μM) was injected into a third channel initially flowing at 100 nl/min. The flow rate was increased to 500 nl/min for 2 minutes in this channel to rapidly equilibrate the inhibitor concentration at the detection point. The flow rate was then reduced to 195 nl/min and a 40-fold continuous concentration gradient was run over 4 minutes from 195 nl/min to 5 nl/min. Assay buffer was added in the fourth channel to maintain total flow rate, which is the sum of all four channels, at 400 nl/min. The inhibitor was flushed out of the system with 2% (v/v) methanol in water. For inhibitor 1, a 3-fold dilution, in assay buffer, of the 6 μM inhibitor was then performed, to give 2 μM inhibitor, which solution was then injected into the third channel and the aforementioned concentration gradient was applied again. The 2 μM inhibitor then underwent a further 3-fold dilution to 670 nM, and the concentration gradient was repeated a third time. For inhibitor 2, a 4-fold dilution, in assay buffer, of the 20 μM inhibitor was then performed, to give 5 μM inhibitor, which solution was then injected into the third channel and the aforementioned concentration gradient was applied again.

The three (for inhibitor 1) or two (for inhibitor 2) continuous gradients were used to generate an IC₅₀ curve and the concentrations of each gradient overlapped to aid matching of the gradient data. The data collected are illustrated in FIGS. 8 and 9, which show the data for inhibitors 1 and 2, respectively.

In a microtitre plate version of the assay, 142 of 1089 inhibitors/compounds screened were found to have IC₅₀ values of >10 μM, which inhibitors/compounds would not usually be pursued further for drug discovery. Using the subject matter disclosed herein, such inhibitors/compounds may only be subjected to one concentration gradient.

The 384-well microtitre plate MMP12 assay screened each inhibitor/compound at 11 concentrations, in duplicate, using a total volume of 51 μl (comprising 1 μl compound, 25 μl enzyme and 25 μl substrate). Thus, using the microtitre plate version, each inhibitor/compound IC₅₀ curve requires 550 μl of MMP12 and 550 μl of substrate.

However, using the subject matter disclosed herein, 8 82 l of enzyme and 8 μl of substrate held in the reservoir loops lasted for approximately 60 mins. Repeat injections of compound were performed every 10 minutes (this being the sample time), which 10 minutes includes the 2 minute concentration gradient. Thus each 40-fold gradient uses ˜1.3 pl of substrate and ˜1.3 μl of enzyme. Therefore, for each inhibitor/compound of >10 μM IC₅₀, if only one continuous concentration gradient is performed, the present subject matter disclosed herein will provide a 413-fold saving in reagent.

Even when three, 40-fold gradients are used to determine an IC₅₀ value, the subject matter disclosed herein provides a 138-fold saving in reagent over the microtitre plate method.

For a 4 minute sample time, the reagent savings increase to 1031-fold and 344-fold, when one and three concentration gradient steps are used, respectively.

For a 2 minute sample time, the reagent savings are further increased to 2063-fold and 688-fold, when one and three concentration gradient steps are used, respectively.

The following table illustrates the reagent savings of the subject matter disclosed herein over said typical microtitre plate based assay.

| | Assay format | | | |
|---|---|---|---|---|
| | Volume of reagent per IC₅₀ (μl) | | Reagent saving per IC₅₀ determination | |
| | determination (this is the sum of | | for embodiments of the subject matter | |
| | the substrate and enzyme volumes) | | disclosed herein compared to the plate assay | |
| | 384-well plate | | | |
| | 1100 | | N/A | N/A |
| Subject matter | One 40-fold | Three 40-Fold | One 40-fold | Three 40-Fold |
| disclosed herein: | Gradient | Gradients | Gradient | Gradients |
| 10 min Sample Time | 2.667 | 8.00 | 413-fold | 138-fold |
| 4 min Sample Time | 1.067 | 3.20 | 1031-fold | 344-fold |
| 2 min Sample Time | 0.533 | 1.60 | 2063-fold | 688-fold |

### Example 2

The FP system has been successfully used in accordance with the subject matter disclosed herein to perform an assay for activin receptor-like kinase 5 (ALK5).

Specifically, a fluorescence polarisation ligand-binding assay for ALK5 ser/thr kinase inhibitors was used. Inhibition was measured by examining the displacement from the enzyme of a fluorescently-labelled ligand by the inhibitor under test. The displacement causes the polarisation value to decrease. The assay used human GST-ALK5 (residues 198-503) expressed in a baculovirus/Sf9 system and a rhodamine green (RhGr) labelled ligand. The ligand and enzyme were prepared to the required concentrations in a 2× assay buffer which consisted of: 125 mM HEPES (pH 7.5), 25 mM MgCl₂, 2.5 mM CHAPS, 0.04% (w/v) sodium azide, 2 mM dithiothreitol (DTT, added just prior to use) in MilliQ purified water [all buffer reagents were from Sigma-Aldrich, except HEPES which was from Invitrogen].

A small molecule inhibitor was used, with an IC₅₀ value known to be approximately 30 nM (at a fluorescently-labelled ligand concentration of 4 nM and an ALK5 concentration of 40 nM) from the microtitre plate-based ALK5 assay. The inhibitor was diluted with 2% (v/v) methanol in water to the required concentration from a 10 mM stock prepared in neat DMSO.

Initially the pump continuously flowed 2% (v/v) methanol in water, which constituted the mobile phase for the assay system, through all four channels. The reagents and the inhibitor were introduced into the system, replacing the mobile phase. The total flow rate in the system was maintained at 400 nl/min. The reaction was performed at 37° C. Prior to injection, the enzyme-ligand complex and the inhibitor were stored at 4° C. in 96-well polypropylene, U-bottomed, clear microtitre plates in a cooled tray on the CTC autosampler. ALK5 is not stable on its own and must therefore be prepared as a complex with the ligand in 2× assay buffer. First, the 2× assay buffer was injected into one channel flowing at 100 nl/min. 4 nM RhGr-labelled ligand/40 nM ALK5 complex was then injected into a second channel flowing at 100 nl/min. The injection syringe was cleaned in 2% (w/v) LDS, 100% (v/v) methanol and water as in example 1. The flow rate was increased in the enzyme-ligand complex channel to 500 nl/min for 3 minutes to quickly equilibrate the concentrations at the detection point. The final concentrations in the assay were: 1 nM RhGr-labelled ligand, 4 nM ALK5, 62.5 mM HEPES (pH 7.5), 12.5 mM MgCl₂, 1.25 mM CHAPS, 1 mM DTT and 0.02% (w/v) sodium azide. Once a stable enzyme-ligand (EL) signal was achieved, the highest concentration of inhibitor (20 μM) was injected into a third channel initially flowing at 100 nl/min. Its flow rate was increased to 500 nl/min for 2 minutes in this channel to rapidly equilibrate the inhibitor concentration at the detection point. Its flow rate was then reduced to 195 nl/min and a 40-fold continuous concentration gradient was applied over 2 minutes. Specifically, the gradient ran from 195 nl/min (9.75 μM inhibitor) to 5 nl/min (250 nM inhibitor). 2% (v/v) methanol in water was added in the fourth channel to maintain total flow rate in all four channels at 400 nl/min. After this, the inhibitor was flushed out of the system with 2% (v/v) methanol in water. A 35-fold dilution, in 2% (v/v) methanol in water, of the 20 μM inhibitor was performed, to give 571 nM inhibitor. This was then injected into the third channel and the concentration gradient was repeated. The 571 nM inhibitor then underwent a further 35-fold dilution and the concentration gradient was repeated a third time.

As in example 1, the three continuous gradients were used to generate an IC₅₀ curve. The overlap in the concentrations of each gradient assisted the matching of the gradient data.

### Example 3

The FL system has been successfully used in accordance with the subject matter disclosed herein to perform an assay for glycogen synthase 3 kinase (GSK3).

An in-house 96 well microtitre plate fluorescence polarisation ligand-binding assay for inhibitors of the protein drug target GSK3 kinase was adapted for use on the FL system. The inhibition was measured by examining the displacement from the enzyme of a fluorescently-labelled ligand by the test inhibitor. The displacement causes a change in lifetime of the fluorophore between its bound and unbound states, which is then measured.

The assay consisted of a human recombinant GSK3β long truncate (i.e. part of the enzyme that is not the full form and contains the active site) expressed in a baculovirus system [in-house] and a Cy5-labelled ligand (with a lifetime=0.8 ns). The ligand and enzyme were prepared to the required concentrations in a 2× assay buffer containing: 100 mM HEPES (pH 7.5), 20 mM MgCl₂, 2 mM CHAPS, 0.04% (w/v) sodium azide and 2 mM DTT (added just prior to use) in MilliQ purified water [all buffer reagents were from Sigma, except HEPES which was from Invitrogen].

A small molecule inhibitor was used, with a Kᵢ value known to be approximately 85 nM and an IC₅₀ value known to be approximately 30 nM, at a ligand concentration of 2 nM and GSK3 concentration of 3 nM, from a microtitre plate-based GSK3 assay. The inhibitor was diluted with 2% (v/v) methanol in water to the required concentration from a 10 mM stock prepared in neat DMSO.

Initially the pump continuously flowed 2% (v/v) methanol in water through all four channels, which constituted the mobile phase for the assay system. The reagents and the inhibitor were introduced into the system, replacing the mobile phase. The total flow rate in the system was maintained at 400 nl/min. The reaction was performed at 37° C. Prior to injection, the enzyme-ligand complex and the inhibitor were stored at 4° C. in glass vials in a cooled tray on the CTC autosampler.

The ligand and enzyme were prepared together as a mix in 2× assay buffer. First, 2× assay buffer was injected into one channel flowing at 100 nl/min. 8 nM Cy5-labelled ligand/12 nM GSK3β complex was then injected into a second channel flowing at 100 nl/min. The injection syringe was then cleaned in 2% (w/v) LDS, 100% (v/v) methanol and water as in examples 1 and 2.

The flow rate was increased in the enzyme-ligand complex channel to 500 nl/min for 3 minutes to quickly equilibrate the concentrations at the detection point. The final concentrations in the assay were: 2 nM Cy5-labelled ligand, 3 nM GSK3β, 50 mM HEPES (pH 7.5), 10 mM MgCl₂, 1 mM CHAPS, 1 mM DTT and 0.02% (w/v) sodium azide. Once a stable enzyme-ligand (EL) signal was achieved, the highest concentration of the inhibitor (20 μM) was injected into a third channel initially flowing at 100 nl/min. Its flow rate was increased to 500 nl/min for 2 minutes in this channel to rapidly equilibrate the inhibitor concentration at the detection point. Its flow rate was then reduced to 195 nl/min and a 40-fold continuous concentration gradient was applied over 2 minutes. Specifically, the gradient ran from 195 nl/min (9.75 μM inhibitor) to 5 nl/min (250 nM inhibitor). 2% (v/v) methanol in water was added in the fourth channel to maintain total flow rate in all four channels at 400 nl/min. After this, the inhibitor was flushed out of the system with 2% (v/v) methanol in water. A 35-fold dilution, in 2% (v/v) methanol in water, of the 20 μM inhibitor was performed, to give 571 nM inhibitor. This was then injected into the third channel and the concentration gradient was repeated. The 571 nM inhibitor then underwent a further 35-fold dilution and the concentration gradient was repeated a third time.

As in examples 1 and 2, the three continuous gradients were used to generate an overall sigmoid curve spanning the entire array of concentrations tested. From this the IC₅₀ value was determined. The overlap in the concentration ranges aided the overlaying of the data from each step.

It will be understood that various details of the subject matter can be changed without departing from the scope of the subject matter. Furthermore, the foregoing description is for the purpose of illustration only, and not for the purpose of limitation.

1. A method for determining a value that allows the effect that a compound has on a target to be compared with the effect that another compound has on the target, which method comprises adding the compound, at a concentration which continuously varies with time, to a flow of the target.

2. A method according to claim 1 wherein the value is the ICₓ value or ECₓ value of the compound, wherein x is a percentage of the target activity.

3. A method according to claim 1 wherein the continuous variation of the compound concentration with time is achieved by keeping the overall flow rate constant whilst changing the flow rate of the compound and/or the target.

4. A method according to claim 3 wherein the continuous variation of the compound concentration with time is achieved by changing the flow rate of the compound whilst changing the flow rate of a component other than the target so as to keep the overall flow rate constant.

5. A method according to claim 1 comprising more than one step of adding the compound to a flow of the target and wherein the concentration of the compound varies continuously with time throughout each of these steps and wherein the rate of change of compound concentration with respect to time in each step is different from the rate of change of compound concentration with respect to time in any of the other steps and/or the compound concentration range in each step is different from the compound concentration range in any of the other steps.

6. A method according to claim 5 in which, within each step, the rate of change of compound concentration with respect to time is constant.

7. A method according to claim 5 in which the rate of change of compound concentration with time differs between each step.

8. A method according to claim 5 in which the rate of change of compound concentration with time is the same in each step.

9. A method according to claim 5 in which:
the difference between the compound concentration at the beginning and end of the step, divided by the compound concentration at the beginning of the step is the same in each step.

10. A method according to claim 5 in which each step is performed for the same length of time.

11. A method according to claim 5 in which:
the difference between the compound concentration at the beginning and end of the step, divided by the compound concentration at the beginning of the step is the same in each step; and
each step is performed for the same length of time.

12. A method according to claim 5 in which the compound concentration range in each step is different from the compound concentration range in (any of) the other step(s).

13. A method for determining a value that allows the effect that a compound has on a target to be compared with the effect that another compound has on the target, which method comprises more than one step of:
adding the compound, at different concentrations, to a flow of a target, wherein the compound concentration range in each step differs from the compound concentration range in any of the other steps.

14. A method according to claim 13 wherein the value is the ICₓ value or ECₓ value of a compound, wherein x is a percentage of the target activity.

15. A method according to claim 13, wherein the highest compound concentration, lowest compound concentration or highest and lowest compound concentrations in each step differ(s) from the highest compound concentration, lowest compound concentration or highest and lowest compound concentrations, respectively, in any of the other steps.

16. A method according to claim 15, wherein the highest and lowest concentrations in each step differ from the highest and lowest concentrations, respectively, in any of the other steps.

17. A method according to claim 5 in which there are three or more steps.

18. A method according to claim 5 in which there is overlap between the ranges used in at least two of the steps.

19. A method according to claim 18 in which the range in each step overlaps with the range in at least one other step.

20-24. (canceled)

25. A method for determining values for more than one compound, wherein each value is associated with one compound and allows the effect that said compound has on a target to be compared with the effect that another compound has on the target, wherein said values may optionally be ICₓ values or ECₓ values, wherein x is a percentage of the target activity, in which each compound is tested using the method of claim 1.

26-51. (canceled)

52. An apparatus adapted to be used to determine a value that allows the effect that a compound has on a target to be compared with the effect that another compound has on the target, which apparatus allows the addition of the compound at a concentration that continuously varies with time to a flow of the target.

53-60. (canceled)
